(19) United States
(12) Patent Application Publication
(10) Pub. No.: US 2020/0197572 A1
(43) Pub. Date: Jun. 25, 2020

(54) THREE-DIMENSIONAL (3D) TISSUE-LIKE IMPLANT AND PREPARATION AND APPLICATION THEREOF

(71) Applicant: National Health Research Institutes, Zhunan Town (TW)

(72) Inventors: Feng-Huei LIN, Miaoli County (TW); Jui-Sheng SUN, Taipei City (TW); Ching-Yun CHEN, Miaoli County (TW); Chih-Ying CHI, Miaoli County (TW)

(73) Assignee: National Health Research Institutes, Zhunan Town (TW)

(21) Appl. No.: 16/231,769

(22) Filed: Dec. 24, 2018

(51) Int. Cl. A61L 27/38; C12N 5/00; C12N 5/0775

(52) U.S. Cl. CPC A61L 27/3847; C12N 5/0062; C12N 2533/90; C12N 2533/74; C12N 5/0663

(57) ABSTRACT

The present invention relates to a three-dimensional (3D) tissue-like implant for transplanting to a subject in need comprising a cell cluster comprising mesenchymal stem cells (MSCs) and specific cells differentiated therefrom. The present invention also relate to a method of preparing a 3D-tissue-like implant from MSCs, particularly by seeding MSCs in alginate scaffolds and culturing the alginate scaffolds with MSCs in a 3-D perfusion condition. Further, the present invention provides a method for treating a defect in a recipient patient in need by administering a 3D tissue-like implant as described herein to the patient at a defective site e.g. a bone defective site.

### TECHNOLOGY FIELD

The present invention relates to a three-dimensional (3D) tissue-like implant for transplanting to a subject in need comprising a cell cluster comprising mesenchymal stem cells (MSCs) and specific cells differentiated therefrom. The present invention also relate to a method of preparing a 3D-tissue-like implant from MSCs, particularly by seeding MSCs in alginate scaffolds and culturing the alginate scaffolds with MSCs in a3D perfusion condition. Further, the present invention provides a method for treating a defect in a recipient patient in need by administering a 3D tissue-like implant as described herein to the patient at a defective site e.g. a bone defective site.

### BACKGROUND OF THE INVENTION

The loss or failure of an organ or tissue is a very severe human health problem. Tissue engineering (TE) is an interdisciplinary field that combines the principles of engineering and biosciences with the goal of achieving human tissue regeneration or reconstruction [1-3]. TE aims at developing engineered tissues or substitutes created in vitro that restore, maintain or improve tissue function [4-6]. It is known that because the differentiation of cells is greatly influenced by the niche that harbors undifferentiated precursors and by both intrinsic and extrinsic signals, a 2D culture approach presents critical limitations resulting in low differentiation efficiency [13]. However, most techniques for investigating mechanisms controlling cell behavior in vitro have been developed using 2D cell culture systems and are of limited use in 3D environments, such as engineered tissue constructs. The biasing of cell function that occurs with traditional methods of 2D culture, leads to unpredictable in vivo results that hamper translation into the clinic.

In particular, a worldwide life expectancy increases annually, age-related skeletal diseases e.g. bone loss are becoming a serious health concerns in almost every population [7, 8]. Regeneration of bone defects remains one of the most significant challenges faced in reconstructive surgery [9]. Considering that spontaneous bone regeneration is limited to relatively small defects, bone graft material is often required for the treatment of large bone defects caused by traumatic injury, osteomyelitis, tumor removal or implant loosening [10, 11]. However, owing to limitations and risks associated with autologous as well as allogenic bone grafting procedures, alternative strategies are required. Recent ex vivo TE strategies for de novo generation of bone tissue include the combined use of autologous bone-forming cells and three-dimensional (3D) porous scaffold materials serving as structural support for the cells. In this regard, bioreactor systems have become key components of bone TE strategies by providing physical stimulation of tissue-engineered constructs and by allowing mass transport to and from the cells. A culture system where osteoblasts are seeded in calcium-alginate scaffolds and cultured in a closed perfusion bioreactor has been reported to generate bone cell clusters for autologous transplantation [31]. However, the source of adult osteoblasts is limited, and they must be obtained by surgery that is painful for patients. Further, the osteoblasts are terminally differentiated cells and thus the problems of cell death remain.

MSC is a specific cell population with highly regulated self-renewing ability; MSCs secrete a wide spectrum of bioactive molecules, including growth factors and cytokines, to avoid allogenic rejection, thus. MSCs can be considered as ideal cell source for therapeutic use and open new frontiers in medicine [28]. The secreted bioactive factors offer a regenerative microenvironment for defect sites to restrict the area of damage and to regenerate native tissues by self-regulating. The adult MSC is culture-dish adherent, so it can be easily isolated from bone marrow aspirates and be expanded in culture while preserving its multipotency. Duo to MSCs had been largely used in preclinical trials and clinical practice for tissue engineering; MSCs, which serve as tissue-engineered materials, hold considerable promise for therapeutic use in repairing and in reconstructing damaged or diseased mesenchymal tissues [29].

MSCs have been used in the tissue engineering technique where MSCs are differentiated and proliferated in vitro in 2D condition for a period of time to generate a sufficient amount of differentiated cells and after enzymatic treatment, a certain amount of the differentiated cells in a free form can be collected. Such free (differentiated) cells are then either directly transplanted into patients, or firstly attached onto proper scaffolds (with pores to increase the surface area for cell growth), cultured in a proper bioreactor for a period of time to achieve a required amount of cells and the cells with scaffolds are finally transplanted into patients [45]. However, differentiated cells in a free form cannot be well fixed and maintained in the defect sites within the body; and even if the differentiated cells are attached to scaffolds, a suitable microenvironment seems not be generated for cell growth or function after moving into the bodies since a high cell death rate is still observed [2]. Further, the above-mentioned approach is not easy to reach a required number of cells due to the limitation of 2D environment, which takes numerous steps and much time to complete. For example, it takes about 6-7 weeks to complete the steps of proliferating and differentiating MSCs in a 2D culture condition, attaching the differentiated cells onto scaffolds, transferring the cells with scaffolds into a bioreactor, and achieving the desired number of cells [46-49]. Moreover, scaffolds may induce inflammatory reactions in the bodies, resulting in prolonged healing time e.g. about 2 months as previously reported [50-51]. In addition to the above, some other approaches have been reported where after MSCs attached onto scaffolds are transferred into defect sites, certain stimulators are given therein in order to generate a suitable microenvironment for the cells to grow and differentiate. However, such approach could be dangerous because MSCs are sensitive to the environment they stay and a variety of undesired cells could be generated when MSCs are exposed to numerous stimulators without suitable protection [52]. On the other hand, extracellular matrix (ECM) is known to be important to the adhesion, proliferation and differentiation of cells, while routine cell detaching/harvest processes, especially via enzymatic (e.g. trypsin) treatment, result in damages to ECM and thus a suitable microenvironment for cells cannot be well established.

### SUMMARY OF THE INVENTION

In this invention, it is unexpectedly found that seeding MSCs in alginate-based scaffolds and in vitro culturing the alginate scaffolds with MSCs in a perfusion bioreactor under a condition that allows proliferation and differentiate of the MSCs toward one or more types of specific cells can generate a three-dimensional (3D) tissue-like implant containing the MSCs and the specific cells in a form of a cell cluster which is useful for transplanting into a subject in need.

Therefore, in one aspect, the present invention provides a method of preparing a 3D tissue-like implant, comprising

(a) seeding MSCs in an alginate scaffold to give a MSCs-alginate construct;

(b) transferring the MSCs-alginate construct into a perfusion bioreactor system; and

(c) incubating the MSCs-alginate construct in the perfusion bioreactor system under a condition that allows proliferation and differentiation of the MSCs toward the specific cells and formation of the 3D tissue -like implant which comprises the alginate scaffold embedded with a cell cluster comprising the MSCs and the specific cells.

In some embodiments, the present invention further comprises (c)′ exposing the 3D tissue-like implant to a chelating agent to dissolve the scaffold to provide a scaffold-free 3D tissue -like implant. The present invention can further comprises (d) collecting the 3D tissue-like implant, far example, by centrifugation.

The present invention further provides a 3D tissue-like implant or a pharmaceutical composition for transplanting into a subject in need, comprising a cell cluster comprising MSCs and specific cells differentiated therefrom, and optionally a pharmaceutically acceptable carrier. In some embodiments, the cell cluster as an active ingredient is formulated with a pharmaceutically acceptable carrier at an amount effective to repair the defect in a subject in need.

The present invention also provides a 3D tissue-like implant for transplanting into a subject in need prepared by a method as described herein.

Specifically, the cell cluster further comprises extracellular matrix surrounding and supporting the MSCs and the specific cells. In some embodiments, the specific cells differentiated from MSCs can be osteo-like cells, chondro-like cells, muscle-like cells, neuron-like cells, adipo-like cells, hepato-like cells, lung-like cells, cardiac-like cells, fibroblast-like cells, and any combination of the above. In some embodiments, the cell cluster forms bone-like, cartilage-like, muscle-like, nerve-like, adipose-like, liver-like, lung-like, heart-like and/or blood vessels-like tissues.

In another aspect, the present invention provides a method for treating a defect in a recipient patient in need, comprising placing a 3D-tissue-like implant or a pharmaceutical composition as described herein to the patient at a defective site at an amount effective to treat the defect.

In particular, the present invention provides a method for repairing a bone defect in a recipient patient in need, comprising
(i) providing a 3D bone-like implant which is prepared by a method comprising (a) seeding MSCs in an alginate scaffold to give a MSCs-alginate construct: (b) transferring the MSCs-alginate construct into a perfusion bioreactor system for cultivation under a condition that allows proliferation and differentiate of the MSCs toward osteo-like cells and formation of the 3D bone-like implant comprising a cell cluster comprising the MSCs and the osteo-like cells; (c) optionally exposing the 3D bone-like implant to a chelating agent to dissolve the scaffold to provide a scaffold-free 3D bone-like implant; and (d) collecting the 3D bone-like implant; and
(ii) placing the 3D-bone like implant to the patient at a bone defective site at an amount effective to repair the bone defect.

Also provided is use of a cell duster or a 3D tissue-like implant as described herein for manufacturing a medicament for treating a defect in a recipient patient in need.

The details of one or more embodiments of the invention are set forth in the description below. Other features or advantages of the present invention will be apparent from the following detailed description of several embodiments, and also from the appending claims.

### BRIEF DESCRIPTION OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention Is not limited to the precise arrangements and instrumentalities shown.

In the drawings:

FIG. 1 shows a particular embodiment of a method to prepare a bone-like tissue according to the present invention.

FIG. 2 shows one embodiment of the bioreactor system as used in the present invention, optionally with a regulator to monitor the culture condition.

FIG. 3 shows the cell surface markers screening and the differential assay of human mesenchymal stem cells. Upper parts: the flow cytometry evaluated the cell surface markers: CD29, CD44, CD73, and CD90 were positive; on the contrary, CD34 and CD45 presented negative. Lower parts: the differential capability: (left) hMSCs differentiated into osteo-like cells in 14 days, and the stained biological apatite: (middle) hMSCs under pellet culture treatment differentiated into chondro-like cells in 21 days, and the stained glycosaminoglecan; (right) hMSCs differentiated into adipo-like cells in 14 days, and the stained lipid droplets.

FIG. 4 shows the live/dead staining results of the hMSCs in alginate scaffolds w/perfusion. Upper parts: (the first row) the live cells of bone-like tissues with calcein AM dye; (the second row) the dead cells; (the third row) the merge images. Lower parts: the percentage of live and dead cells.

FIG. 5 shows the results of apoptotic detection of the bone-like tissues in alginate scaffolds cultured in the bioreactor system according to the present invention. Upper parts: (the first row) activated caspase 3/7 indicated the apoptotic cells of bone-like tissues; (the second row) the stained nucleus; and (the third row) were the merge images. Lower parts: the level of activated caspase 3/7.

FIG. 6 shows the results of the mitochondrial transmembrane potential detection of the bone-like tissues in alginate scaffolds cultured in the bioreactor system according to the present invention. Upper parts: (the first row) JC-1 monomer indicated the apoptotic cells of bone-like tissues; (the second row) JC-1 aggregates represented the healthy cells of bone-like tissues; and (the third row) the merge images. Lower parts: the levels of JC-1 monomer (damaged cells) and JC-1 aggregates (healthy cells).

FIG. 7 shows the structure and mitochondrial mass of bone-like tissues in alginate scaffolds cultured in the bioreactor system according to the present invention. Upper parts: (the first row) the structure of bone-like tissues with the nucleus; (the second row) the mitochondrial mass of bone-like tissues with the nucleus; (the third row) the merge images. Lower parts: the level of mitochondrial mass.

FIG. 8 shows the morphology of bone-like tissues in alginate scaffolds cultured in the bioreactor system according to the present invention. Upper parts: (the first row) the morphology of hMSCs in alginate scaffolds were examined by SEM under 500× observation; (the second flow) the images were under 2000× observation. Lower parts: the EDX determination showed calcium and phosphorous ions increased over time.

FIG. 9 shows the results of the evaluation of endochondral ossification in the bioreactor system. Upper parts: (the first row) the cross-sectional view of the images of Live/Dead staining (FIG. 7); (the second row, left) the sGAG levels in the culture media; (the second row, right) the measurement of ALP activity from the culture media. Lower parts: (the first row) Safranin O represented the production of GAGs from bone-like tissues in the bioreactor system; (the second row) Xylenol Orange revealed the biomineralized area of bone-like tissues in the bioreactor system.

FIG. 10 shows the results of micro-CT and the determination of ICP-OES. (Upper parts) showed the process of biomineralization via micro-CT evaluation, the cells/scaffolds constructs were getting harder through the time; (Middle parts) the relative vBMD, which was mean±SD (▴p<0.05 vs. Day 1 group; * p<0.05 vs. Day 7 group: #p<0.05 vs. Day 14 group; +p<0.05 vs. Day 21 group, n=3). (Lower parts, left) presented the calcium ion concentration of cell culture remains in the specific incubation period; (Lower parts, right) represented the phosphorus ion concentration of cell culture remains in the specific incubation period.

FIG. 11 shows the patterns of XRD and FT-IR (Left) showed the XRD pattern; (Right) presented the FT-IR data.

FIG. 12A-12D shows the results of bone-related mRNA expression of the bone-like tissues in alginate scaffolds cultured in the bioreactor system according to the present invention. For Ctrl group, undifferentiated hMSCs were cultured in 2D condition without osteogenic induction. After 7, 14, 21 and 28 days perfusion, the bone-like tissues were collected for gene expression examination. Expression of (FIG. 12A) CD73, CD90 and CD105; (FIG. 12B) ALP, RUNX2 and OCN; (FIG. 12C) OPN, BMP-2 and VEGF-A; (FIG. 12D) Col1A1, Col2A1 and MMP-3, were analyzed via Q-PCR protocols. The relative mRNA level was calculated following 2^{-ΔαCt} method, and each target gene was normalized to Ctrl group. The Q-PCR values were mean±SD (▴p<0.05 vs. Day 1 group; *p<0.05 vs. Day 7 group; #p<0.05 vs. Day 14 group; +p<0.05 vs. Day 21 group; n=6).

FIG. 13A-13B shows the results of the expression of growth factors and bone-related proteins secreted from bone-like tissues in alginate scaffolds cultured in the bioreactor system according to the present invention. After 7, 14, 21 and 28 days' perfusion, the culture media were collected for ELISA examination. Expression of (FIG. 13A) TGF-β1, OCN, OPG and BMP-2; and (FIG. 13B) sCD105, bfGF. SDF-1α and VEGF, were analyzed via manufacturer's guidelines. The data were mean±SD (▴p<0.05 vs. Day 1 group; *p<0.05 vs. Day 7 group; #p<0.05 vs. Day 14 group; +p<0.05 vs. Day 21 group; n=6).

FIG. 14 shows the results of live/dead staining of the bone-like tissues in alginate scaffolds cultured in the bioreactor system according to the present invention. Upper parts: (first row) represented the live cells of bone-like tissues with calcein AM dye; (second row) indicated dead cells; (third row) were the merge images. Lower parts: displayed the percentage of live and dead cells.

FIG. 15 shows the results of live/dead staining showed the difference between hMSCs @Ca-Alginate scaffolds in static xeno-free system. Upper parts: (first row) represented the live cells of bone-like tissues with calcein AM dye; (second row) indicated dead cells; (third row) were the merge images. Lower parts: displayed the percentage of live and dead cells.

FIG. 16 shows the results of the examination of micro-CT and the determination of XO staining. Upper pans: the relative vBMD was presented as mean±SD (n=3). Lower parts: (first row) show-ed the process of biomineralization via micro-CT evaluation, the cells/scaffolds constructs were getting harder through the time; and (second row) XO revealed the biomineralized area of bone-like tissues in the bioreactor system, and the stained nucleus.

FIG. 17 shows the results of the in-vivo animal study. (Upper parts) showed the subcutaneous engraftment in NOD/SCID mice (Sham: Sham group, PBS injection; NC: negative control group, clinical-grade type I collagen solution injection; D14MT: 1^{st} experimental group, clinical-grade type I collagen solution combining with the bone-like tissues for 14-day's perfusion; D21MT: 2^{nd} experimental group, clinical-grade type I collagen solution combining with the bone-like tissues for 21-day's perfusion. (Lower parts) revealed the relative vBMD, which was mean±SD (▴p<0.05 vs. Sham group; * p<0.05 vs. NC group; #p<0.05 vs. D14MT group; +p<0.05 vs. Day 21 group, n=3).

FIG. 18A-18C shows the results of the micro-CT evaluation for in-vivo engraftment test at specified time periods, Day 1 (FIG. 18A), Week 2 (FIG. 18B) and Week 4 (FIG. 18C).

Unless defined otherwise, all technical and scientific terms used herein have the same meaning as commonly understood by a person skilled in the art to which this invention belongs.

1. Definitions

As used herein, the singular forms “a”, “an”, and “the” include plural referents unless the context clearly dictates otherwise. Thus, for example, reference to “a component” includes a plurality of such components and equivalents thereof known to those skilled in the art.

The term “comprise” or “comprising” is generally used in the sense of include/including which means permitting the presence of one or more features, ingredients or components. The term “comprise” or “comprising” encompasses the term “consists” or “consisting of.”

The term “about” as used herein means plus or minus 10% of the numerical value of the number with which it is being used. Therefore, about 1% means in the range of 0.9% to 1.1%.

The term “substantially” as used herein refers to a majority of, or mostly, as in at least about 50%, 60%, 70%, 80%, 90%, 95%, 96%, 97%, 98%, 99%, or 100%.

As used herein, the term “three-dimensional (3D) tissue-like implant” includes a mass of cells functionally bound to each other, forming a cell cluster, capable of no longer responding only individually but also present like a functional tissue or organ, which is useful as an implant for transplanting into a subject in need thereof. In particular, a cell duster as described herein is a “3D” cell cluster (or aggregate or mass) which is different from a 2D cell culture (e.g. a monolayer or few layers of cells attached to the surface of a scaffold for cell growth) at least in that it contains more than a few layers of cells and more particularly it forms a sterically structure and morphology. Specifically, a 3D cell cluster can include an extracellular matrix (ECM), which is a network of proteins (such as fibronectin, laminin, collagens and vitronectin), carbohydrates (such as glycosaminoglycans) and other components, forming a scaffold surrounding the cells, like a physical microenvironment in which cells exist, providing (structural/functional) support and connection between cells. A 3D cell cluster as described herein may contain one type of cell or may contain a plurality of different types of cells. For instance, a 3D cell cluster as described herein can contain ostero-like cells including bone-progenitor cells or more mature (terminally differentiated) bone cells e.g. osteoblasts, osteoclasts, and osteocytes; these cells aggregate together with ECM, forming a bone-like tissue. In some embodiments, a 3D cell cluster is a spherical or spherical-like cell cluster, having a diameter of 5 μm to 500 μm, for example, particularly 10 μm to 400 μm more particularly 20 μm to 300 μm. A 3D cell cluster can be easily collected through filtration or centrifugation.

As used herein, the term “mesenchymal stem cells (MSCs)” refer to multipotent stem cells that can differentiate into a variety of cell types such as osteoblasts (bone cells), chondrocytes (cartilage cells), muscle cells, neuron cells, adipocytes (fat cells), hepatocytcs (liver cells), lung cells, cardiac cells and fibroblasts. MSCs can be obtained from various tissues, such as bone marrow, adipose tissue, muscle tissue, dental tissues, placenta, umbilical cord tissue, umbilical cord blood and peripheral blood. In one embodiment, MSCs are obtained from bone marrow using standard procedures known in the art.

As used herein, the term “multipotency” herein refers to a stem cell that has the ability to differentiate into more than one cell types. A multipotent stem cell can become at least one or two certain cell, type. For example, MSCs can differentiate into osteoblasts, adipocytes, and chondrocytes.

As used herein, the term “differentiation” can refer to a process for differentiating multipotent stem cells (e.g. MSCs) into progeny that are enriched for cells of a particular form or function. Differentiation is a relative process. For example, bone-progenitor cells differentiated from MSCs are relatively primitive when compared to the resultant mature (terminally differentiated) bone cells e.g. osteoblasts, osteoclasts, and ostcocytes.

As used herein, the term “specific cells” can refer to a group of cells that are relatively differentiated from MSCs. Specifically, the term “specific cells” does not include MSCs.

As used herein, the term “proliferation” can refer to growth and division of cells. In some embodiments, the term “proliferation” as used herein with respect to cells refers to a group of cells that can increase in number over a period of time.

As used herein, the term “scaffold(s)” as used herein refers to a matrix or construct e.g. a porous biodegradable polymer that supports cell growth and/or migration, for example.

As used herein, the term “alginate scaffold(s)” refers to a scaffold comprising alginate or alginic acid. Alginic acids are linear polysaccharides comprising repeating units of D-mannuronic acid (M units) and L-gluronic acid (G units). Alginates are salts of alginic acids such as sodium, potassium or ammonium salt, or bivalent calcium or magnesium salt and mixture thereof of alginic acid. Specifically, the alginate or alginic acid cat have a molecular weight of from about 10 kDa to about 600 kDa, preferably about 50 kDa to about 400 kDa; and/or have viscosity of from about 1 centipoise (cP) to about 40,000 cP. preferably about 4 cP to about 10,000 cP.

As used herein, the term “calcium-alginate scaffold(s)” refers to an alginate scaffold that is cross-linked with calcium tons.

As used herein, the term “seeding” refers to plating, placing and/or dropping cells to an environment e.g. a scaffold for culture. For example, the cells (e.g. MSCs) will adhere to the scaffold to form a “cells-alginate construct” (e.g., MSCs-alginate construct) where the cells grow and/or differentiate in the scaffold.

As used herein, the term “bioreactor” refers to a system to culture cells where a biological reaction or conversion occurs to produce one or more desired products for use in for example, tissue engineering or biochemical engineering. In general, a bioreactor provides a closed-loop culture environment where the entry and release of cultivation fluid and/or gas required for cell culture is controlled. In particular, a bioreactor enables dynamical cultivation of cells in a three dimensional environment where cultivation fluid flows around the cells providing nutrients thereto via stirring, rolling or perfusion, for example. Specifically, a perfusion bioreactor can provide gentle and effective transportation of nutrients, oxygen, and waste removal to and from the cells and the core of the scaffold where cells are seeded, e.g. via diffusion, especially cultivation fluid can be uniformly flows without generating undesired shear force causing cell death that is a common problem in a rolling or Stirring bioreactor. More preferably, a perfusion bioreactor system as described herein provides a condition where cultivation fluid flows in a gentle rate such that after a cells-alginate construct is transferred to and incubated in the system, the cells arc not “attached” to the surface of the alginate scaffold in a spread-out, flat morphology as in a conventional 2D culture plate and instead a substantial amount of the cells are kept in non-attached morphology (e.g. a round or oval shape) and stay just around or within the porous structures of the alginate scaffold (without being released out of the scaffold) to perform proliferation and differentiation and then can aggregate to form a cell cluster embedded within the alginate scaffold. The flow rate can be adjusted based on various factors e.g. the cell number/density, the volume of die culture medium and the size of cell culture tank.

As used herein, the term “scam-free” is used to describe a culture and/or a culture medium substantially without scrum or plasma.

As used herein, the term “implant” refers to any object that is designed to be placed partially or wholly within a patient's body for one or more therapeutic or prophylactic purposes such as for restoring physiological function, alleviating symptoms associated with a disease, and/or repairing, replacing, or augmenting damaged or diseased organs and tissues.

2. Three-Dimensional (3D) Tissue-Like Implant and Pharmaceutical Composition

According to the present invention, a 3D tissue-like implant containing certain specific cells can be prepared by seeding MSCs in an alginate scaffold and culturing the resultant MSCs-alginate construct in a perfusion bioreactor system under a condition that allows proliferation and differentiation of the MSCs toward the certain specific cells and formation of the 3D tissue-like implant that comprises the alginate scaffold embedded with a cell cluster comprising the MSCs and the specific cells.

Alginate scaffolds are available and can be prepared by a method known in the art. For example, a free-drying method can be used to prepare the scaffolds, which comprises the following steps: (i) providing an alginate solution, (ii) freezing the alginate solution and subjecting the solution to freeze-drying to generate porous structure, (iii) cross-linking the spongy structure, and (iv) sterilizing and dehydrating the cross-linked spongy structure that can be stored at room temperature until use.

The alginate scaffolds are cross-linked with a crosslinking agent to increase their mechanical strength. In some embodiments, the alginate scaffolds are crosslinked with divalent metal ions (e.g. Ca²+, Ba²⁺, Mg²⁺, Sr²⁺, Zn²⁺).

In some particular embodiments, a calcium solution at a concentration of about 2% to about 15%, e.g. about 2% or higher, about 5% or higher, about 7.5% or higher, and about 10% or higher, up to about 15%, is used to perform the cross-linking reaction.

Suitable scaffolds may have one or more structural features that allows sufficient transportation of media components to cells, removal of wastes from cells and the cells can stably stay around or within the porous structures. Suitable scaffolds may have a porosity of from about 70 to about 95 percent or more. In some embodiments, the scaffolds may have a porosity of from about 80 to about 90 percent or more, more particularly from about 85 to about 95 percent or more. Suitable scaffolds may have an average pore size diameter of from about 50 μm to about 1,000 μm, particularly about 50 μm to about 800 μm.

MSCs can be obtained from various tissues, including but not limited to, bone marrow, adipose tissue, muscle tissue, dental tissues, placenta, umbilical cord tissue, umbilical cord blood and peripheral blood. In certain embodiments, MSCs arc collected from bone marrow aspirate via surgery. The mononuclear cells fraction arc isolated and incubated in suitable medium at 37° C., 5% CO₂. Non-attachcd cells arc removed, leaving attached cells to grow. The MSCs can be expanded for about 3-4 cultivation passages before seeding in the scaffolds.

MSCs are then seeded into the scaffolds to form MSCs-alginate constructs. In some embodiments, MSCs are suspended in culture medium and seeded into the scaffolds at an average density of about 1×10⁵ to about 1×10⁷, particularly about 1×10⁵ to about 2×10⁶ cells per scaffold. After seeding, the cells can be incubated for about 24 hours for adhesion with the scaffolds, and the resultant MSCs-alginate constructs can be directly placed in a perfusion bioreactor for cell culture. Preferably, it takes about 24 hours (1 day), no more than 72-120 hours (3-5 days), for the cell adhesion to the scaffold and then the cell proliferation and differentiation substantially occur in the next stage, i.e. after being transferred into a perfusion bioreactor.

The cell culture in the perfusion bioreactor is carried out under a condition that allows proliferation and differentiation of the MSCs toward specific cells of interest and formation of a 3D tissue-like implant of such specific cells. Specifically, the bioreactor can include a suitable culture medium to perform the cell culture, which comprises a basic medium and additional components to induce differentiation of MSCs toward specific cells of interest as needed. Examples of specific cells of interest include but are not limited to osteo-like cells, chondro-like cells, muscle-like cells, neuron-like cells, adipo-like cells, hepato-like cells, lung-like cells, cardiac-like cells, fibroblast-like cells, and any combination of the above. Such “specific cells” as describe described herein can refer to a group of cells that are relatively differentiated from MSCs which may contain one particular type of cells or may contain several types of cells in various differentiated stage or of different functions in the same lineage. For examples, osteo-like cells can refer to several types of cells in the osteogenic lineage which may include bone-progenitor cells or more mature (terminally differentiated) bone cells e.g. osteoblasts, osteoclasts, and ostcocytes. Culture medium for use in inducing differentiation of MSCs into specific cells of interest can be available in this art.

A basic medium typically contains essential elements for growth and proliferation of the cell including sugars, amino acids, various nutrients, minerals, and the like. Various media are commercially available in the art, for example, including a Dulbecco's modified eagle's medium (DMEM), a minimal essential medium (MEM), and a basal medium eagle (BME). A basic medium can be added with additional components to induce differentiation of MSCs toward specific cells of interest.

In certain embodiments, to induce osteogenic differentiation, a basic medium is supplemented with a corticosteroid (e.g. dexamethasone) and a phosphate source (e.g. ascorbic acid-phosphate and β-glycerophosphate).

In certain embodiments, to induce chondrogenic differentiation, a basic medium is supplemented with insulin and tumor growth factor beta (e.g. TGF-β1, TGF-β2, TGF-β3).

In certain embodiments, to induce adipogenic differentiation, a basic medium is supplemented with a corticosteroid (e.g. dexamethasone), insulin, isobutylmethylxanthine, and indomethacin.

In some embodiments, the basic medium generally can further be supplemented with scrum ingredients (for example, fetal bovine scrum (FBS)), antibiotics (for example, penicillin and streptomycin, and other supplements (for example, pyruvate, insulin, transferrin, selenius acid, and linoleic acid).

In some embodiments, the culture medium as used herein is serum free, and the culture medium instead includes xenogeneic-free/scrum substitutes e.g. UltraGRO. In other embodiments, the culture medium as used herein can contain serum, at a concentration ranging from 5% to 30%, preferably 15% to 25%.

In particular, the perfusion bioreactor system as described herein provides a proper condition suitable for formation of a 3D cell cluster containing the MSCs and the specific cells. Specifically, in the bioreactor system, the culture medium is circulated at a flow rate that provides sufficient supply of nutrition to the cells and regular removal of waste from the cells, and is sufficient to make a substantial amount of the cells exhibit a non-attached (non-spread or non-flat) morphology and stay around or within the porous structures of the alginate scaffold which provides a suitable 3D microenvironment where cell proliferation and differentiation are carried out and then these non-attached cells can grow and aggregate together to form a cell cluster embedded in the alginate scaffold. Preferably, the culture medium is to flow uniformly and consistently without generating undesired shear force causing cell death. The flow rate can be adjusted based on various factors e.g. the cell number/density, the volume of the culture medium and the size of cell culture tank. In some particular embodiments, the flow rate of the culture medium in the perfusion bioreactor system is kept at about 0.001 to about 20 mL/min, particularly at about 0.1 to about 10 mL/min, for example, at about 1 mL/min. In addition, the bioreactor system can provide a normal temperate at about 37° C. and a typical oxygen concentration from about 0.5% to about 21%, for cell culture

Various bioreactor configurations are known and available in this art. In various embodiments, the bioreactor system includes one or more of: a tank to supply culture medium (e.g. a glass bottle), a tank to perform the culture (e.g. a centrifuge tube), one or more pumps (e.g. peristalic pumps) to circulate the medium, a plurality of pipes, control valves, containers, stir blade, and a monitor/regulator unit including one or more detectors or sensors, data processors and monitors. Typically, the bioreactor system as described herein comprises a culture medium tank and a cell culture tank. The culture medium tank contains culture medium and the cell culture tank receives culture medium where the MSCs-alginate constructs can be placed to perform cell culture. Normally, there are a plurality of pipes connected between the two tanks to circulate and transfer the culture medium between them. The system can further comprise a perfusion pump operable to circulate the culture medium in a suitable flow rate. The culture medium tank can further contain ports/openings for gas perfusion and medium exchange. Specifically, the bioreactor system in use allows die culture medium flowing out from the culture medium tank into the culture tank and flowing back out from the culture tank to the culture medium tank, to provide required nutrients and remove wastes for cell growth in a stable manner. The bioreactor system can further comprise a monitor/regulator unit to detect the culture condition at certain time points or perform real-time detection, including the concentrations of oxygen, glucose and nitrogenous waste, and pH. In some embodiments, the bioreactor system can further comprise a container to provide a dissolution agent (e.g. a chelating agent) which can be transported into the culture tank to dissolve the scaffold and thereby a scaffold-free cell cluster product is obtain.

After a suitable period of time for the cell culture in the bioreactor, a 3D tissue-like implant which comprises a cell cluster comprising MSCs and specific cells differentiated therefrom embedded within an alginate scaffold can be produced. In some embodiments, the cell culture can be carried out for at least 1 day or more, 3 days or more, 7 days or more, 14 days or more, 21 days or more, 28 days or more, as needed. Suitable culture medium can be chosen to drive the MSC's differentiation direction to specific cells of interest which are known and available in this art. In some embodiments, specific cells of interest include but are not limited to osteo-like cells, chondro-like cells, muscle-like cells, neuron-like cells, adipo-like cells, hepato-like cells, lung-like cells, cardiac-like cells and fibroblast-like cells. In some embodiments, the cell cluster forms bone-like, cartilage-like, muscle-like, nerve-like, adipose-like, liver-like, lung-like, heart-like and/or blood vessels-like tissues.

The 3D cell cluster as formed according to the present invention can be analyzed and confirmed for their features including the morphology and cell types. The method of the present invention can include steps to perform routine assays to confirm one or more features of the 3D tissue-like implant as prepared, for example, electron microscope and immunological staining. A cell marker detection can be used to confirm that the cell cluster displays both a MSC surface marker and a differentiation marker of specific cells. The cell cluster including MSCs and specific cells make it possible to occur both cell proliferation and differentiation and thus enhance cell viability. In some embodiments, a cell viability test demonstrate that the cell cluster contains live cells at a ratio of 50% or more (e.g. 60% or more, 70% or more, 80% or more, 90% or more, 95% or more) based on the total cells in the cell cluster. The cell cluster including MSCs and specific cells according to the present invention are formed in a mimic niche (microenvironment) where the cells are protected and trapped, in close contact with surrounding extracellular matrix and subject to cellular interactions that support normal cell differentiation, proliferation and function.

In one particular example, a 3D tissue-like implant as described herein comprises a cell cluster made of MSCs and osteo-like cells surrounding with extracellular matrix, forming bone-like tissues. Exemplary conditions for the cell culture to obtain the bone-like tissues accordingly include:
The cell culture is performed in an osteogenic medium containing a basic medium, a corticosteroid (e.g. dexamethasone), a reducing agent (e.g. ascorbic acid-phosphate) and an inorganic phosphate source (e.g. β-glycerophosphate).
The cell culture is performed at 37° C. for about 7 to 21 days or longer e.g. for 28 days.
The medium is circulated with a flow rate of about 0.1-10 mL/min with 0.5-21% oxygen.
The ratio of MSC cell number per scaffold is about 1×10⁵ to 2×10⁶ cells per scaffold.
A culture tank contains 1 to 20 scaffolds.

Such bone-like tissues exhibit one or more features as follows:
the cell cluster forms bone-like tissues via endochondral ossification.
the bone-like tissues include both osteogenic and chondrogenic features, die cell cluster surrounds with extracellular matrix (ECM) and/or calcified areas.
the bone-like tissues display increasing volumetric bone mineral density (vBMD) value, increasing calcium tons and/or phosphorous ions, and/or increasing calcified areas overtime during the cultivation.
the bone-like tissues display volumetric bone mineral density (vBMD) value from about 0.03 mg/cm³ to about 0.13 mg/cm³ and/or Ca/P atomic ratio from about 1.85 to about 1.98.
the bone-like tissues include hydroxyapatite (HAp).
the bone-like tissues display a MSC surface marker, a cartilage marker, an osteogenic marker/growth factor and/or an osteogenic cofactor/associated growth factor
   the MSC surface marker is selected from the group consisting of CD73, CD90, CD 105 and any combination thereof.
   the cartilage marker is secreted glycosaminoglycans (sGAG).
   the osteogenic marker/growth factor is selected from the group consisting of alkaline phosphatase (ALP), osteocalcin (OCN); osteoprotegerin (OPG), bone morphogenetic protein-2 (BMP-2), tumor growth factor beta1 (TGFβ1), vascular endothelial growth factor A (VEGF-A) and any combination thereof.
   the osteogenic cofactor/associated growth factor is selected from the group consisting of sCD105, basic fibroblast growth factor (bFGF), stromal cell derived factor-1alpha (SDF-1α). vascular endothelial growth factor (VEGF) and any combination thereof.
the bone-like tissues do not include vascular cells.

After the cell culture is completed, the 3D tissue-like implant as produced can be simply collected, for example, by centrifugation. An enzymatic treatment e.g. trypsinization to detach adherent cells from the culture plate or matrix, is not needed. In such manner, the cells and the extracellular matrix supporting the cells in the cell cluster can be well preserved and the 3D tissue-like implant as produced can be collected from the culture without substantial damages due to conventional enzymatic treatment.

In some embodiments, the 3D tissue-like implant is further exposed to a dissolution agent such as a chelating agent to dissolve die scaffolds so as to provide a scaffold-free 3D tissue-like implant. In some embodiments, the bioreactor system can include a container containing a dissolution agent which can be transferred from the container to the culture tank to dissolve the scaffold and then to provide a scaffold-free 3D tissue like implant. Preferably, such dissolution agent is chosen and used in a proper amount to sufficiently dissolve the scaffold without causing substantial damages to the cells and the extracellular matrix. A dissolution agent can be a chelating agent such as ethylenediminetetra acetic acid (EDTA), sodium citrate or ethyleneglycol-bis (2-aminoethylether)-N′,N′,N′,N′-tetraactic acid (EGTA)

According to the present invention, a tissue-like cell cluster as described herein may be used an active ingredient for treating a defect in a recipient patient in need. In some embodiments, a therapeutically effective amount of the active ingredient may be formulated with a pharmaceutically acceptable carrier into a pharmaceutical composition in an appropriate form for the purpose of delivery and absorption. Depending on the mode of administration, the pharmaceutical composition of the present invention preferably comprises about 0.1% by weight to about 100% by weight of the active ingredient, wherein the percentage by weight is calculated based on the weight of the whole composition. The composition can be used directly as an implant or further modified to a suitable form for transplantation.

As used herein, “pharmaceutically acceptable” means that the carrier is compatible with the active ingredient in the composition, and preferably can stabilize said active ingredient and is safe to the individual receiving the treatment. Examples of a pharmaceutically acceptable carrier include conventional buffers (phosphoric acid, citric acid, other organic acids, etc.), physiological saline, sterilized water, anti-oxidants (ascorbic acid, etc.), isotonic agents, and preservatives.

In some embodiments, the composition according to the present invention is formulated into a dosage form suitable for injection, where the cell cluster is suspended in a pharmaceutically acceptable carrier e.g. sterilized water or physiological saline or frozen for storage before use. In some embodiments, the composition can further comprise a biodegradable polymer which is useful in stabilizing, supporting and fixing the cell cluster after being locally injected into the defective site. A biodegradable polymer can slowly decomposes in the body after a certain period of time and is preferably biocompatible. Example of such biodegradable polymers include, but are not limited to collagen, fibrin, gelatin, chitosan, alginate, hyaluronic acid, dextran, polylactic acid, polyethyleneglycol. polyacrylic acid, and mixtures thereof The composition according to the present invention can be formulated as a unit dosage form or incorporated into a multiple dose container. The dosage forms may be a suspension, solution, or emulsion in oil or aqueous medium, or powders, granules, tablets, or capsules. The composition of the invention may be delivered through a physiologically acceptable route, typically via injection

3. Methods to Repair Tissue Defects

A tissue-like cell cluster as described herein can be transplanted in a recipient subject in need to treat a tissue defect therefor.

Therefore, the present invention provides a method for treating a defect in a recipient patient in need, comprising placing an implant or a pharmaceutical composition comprising a tissue-like cell cluster as described herein to the patient at a defective site at an amount effective to treat the defect The defect to be repaired can include a defect in bone, cartilage, muscle, nerve, adipose, liver, lung, heart and/or blood vessels.

In particular, the method of the present invention is to repair a bone defect in a recipient patient in need, which comprises
(i) providing a 3D bone-like implant which is prepared by a method comprising (a) seeding MSCs in an alginate scaffold to give a MSCs-alginate construct; (b) transferring the MSCs-alginate construct into a perfusion bioreactor system for cultivation under a condition that allows proliferation and differentiate of the MSCs toward osteo-like cells and formation of the 3D bone-like implant comprising a cell cluster comprising the MSCs and the osteo-like cells; (c) optionally exposing the 3D bone-like implant to a chelating agent to dissolve the scaffold to provide a scaffold-free 3D bone-like implant; and (d) collecting the 3D bone-like implant; and
(ii) placing die 3D-bone like implant to the patient at a bone defective site at an amount effective to repair the bone defect.

The term “individual” or “subject” or patient used herein includes human and non-human animals such as companion animals (such as dogs, cats and the like), farm animals (such as cows, sheep, pigs, horses and the like), or laboratory animals (such as rats, mice, guinea pigs and the like).

The term “treating” as used herein can refer to the application or administration of a composition or implant including one or more active agents to a subject afflicted with a disorder, a symptom or conditions of the disorder, or a progression of the disorder, with the purpose to cure, heal, alleviate, relieve, alter, remedy, ameliorate, improve, or affect the disorder, the symptoms or conditions of the disorder, the disabilities induced by the disorder, or the progression of the disorder or the symptom or condition thereof. Specifically, treating a defect site e.g. a bone damage site, includes aiding recovery, regeneration or reversion of the bone from the damaged status toward a normal/healthy status, completely or partially.

The term “effective amount” used herein refers to the amount of an active ingredient to confer a desired therapeutic effect in a treated subject. For example, an effective amount for treating a bone damage site may be an amount of a bone-like tissue as described herein sufficient to cause a certain degree of recovery (or reversion) from the damaged status toward a normal status, completely or partially, the effective amount may change depending on various reasons, such as administration route and frequency, body weight and species of the individual receiving said active ingredient, and purpose of administration. Persons skilled in the art may determine the dosage in each case based on the disclosure herein, established methods, and their own experience.

A routine method can be used to deliver a 3D tissue-like implant as described to a recipient patient in need, for example, by injection via suitable needles into a defective site to be treated.

The present invention is further illustrated by the following examples, which are provided for the purpose of demonstration rather than limitation. Those of skill in the art should, in light of the present disclosure, appreciate that many changes can be made in the specific embodiments which are disclosed and still obtain a like or similar result without departing from the spirit and scope of the invention.

### EXAMPLES

This study presented an exemplified platform, which were composed of hMSCs, alginate scaffolds, and a perfusion bioreactor system, to generate a bone-like tissue for bone autogenic transplantation. The bioreactor is a closed-process perfusion system, which not only offers the basal cell culture media for nutrient transportation, but also prevents cell contamination resulting from media replacement. Moreover, the secreted cytokine and growth factor can work directly and feedback on the cells in this dynamic system. The alginate scaffold in this system supplied as a cell niche for cell in growth, proliferation, differentiation, and function maintenance. Concern for safety, personalized bone-like tissues allow autogenic transplantation without the risk of immune reactions. In addition, the system is easily to assemble and serves as a convenient tool for researchers or medical doctors. Furthermore, all components of the apparatus are disposable, and the price is affordable for patients. Consequently, this strategy can be applied on cell therapy and opens new avenues for surgical interventions to overcome bone disorders.

1. Material and Methods

1.1 Alginate Scaffold Fabrication and Preparation

The Alginate scaffolds were prepared by a freeze-drying technique as described previously [27]. Briefly. 1.5 wt % pharmaceutical-grade sodium alginate (Keltone® LV, FMC BioPolymer) powder was dissolved in deionized water, and injected into 48-well culture plate with the volume of 1 mL/well. The polymer solution was frozen at 20° C. overnight and then fabricated into porous structure by freeze-drying technique. The spongy scaffolds were cross-linked in 2% calcium chloride solution at room temperature for 1 h. then sterilized with 75% alcohol, dehydrated in a gradient series of ethanol and stored at room temperature until use.

1.2 hMSCs Isolation and Expansion

hMSCs were collected from hone marrow aspirate at total hip/knee joint replacement surgery (IRB No. 201112082 R1C). Mononuclear cells (MNC) traction were isolated according to standard techniques by using a sterile density gradient media. Ficoll-Paque PLUS (an aqueous solution of density 1.077±0.001 g/ml, GE Healthcare, UK), and centrifuging around 300×g at 20° C. for 40 min. The isolated cells were washed with PBS for 3 times and resuspended in low glucose Dulbccco's Modified Eagle's medium (LG-DMEM) supplemented with 10% fetal bovine scrum (FBS, Biological Industries, Israel). These cells were cultured at 37° C. in 5% CO₂ atmosphere for 3 days. After 72 h incubation, the non-adherent cells were removed by washing with PBS gently and the adherent cell population were left behind to grow. When reaching 70-80% confluence, the cells were trypsinized and subculturcd for expanding. In this study, the hMSCs were used at passage 3-4 throughout the follow ing experiments.

1.3 Characteristics of hMSCs Analysis by Flow Cytometry (FC)

The immunophenotypic analysis of hMSCs were carried out using direct staining protocols with conjugated monoclonal antibodies using flow cytometry method. The isolated cells of passage 3 were characterized with respect to the expression of surface antigens. The expression of the following four surface antigens: CD29 (BD Biosciences, USA), CD34 (BD Biosciences, USA), CD44 (BD Biosciences, USA). CD45 (BD Biosciences, USA), CD73 (BD Biosciences, USA), and CD90 (BD Biosciences. USA) were characterized confirmed by LSR II flow cytometer with 488 nm laser option (BD Biosciences, USA). The data were analyzed with tire FlowJo software (Treestar, USA). Utilize forward and side scatter (FSC/SSC) profile to distinguish signal cell population and gate out debris or dead cells.

1.4 Differential Assay of hMSCs

To induce osteogenic differentiation, MSCs (passages P0-P2) were seeded at 5×10³ cells/cm² on tissue culture plastic plates and cultured in osteogenic medium. Osteogenic medium consists of low-glucose DMEM (Gibco) supplemented with 2% Fetal bovine scrum (FBS. Biological Industry). 1% penicillin-streptomycin-amphotericin (PSA, Biological Industry), 0.1 μM dexamethasone (Sigma-Aldrich), 0.2 mM L-ascorbic acid 2-phosphate (Sigma-Aldrich), and 10 mM β-glycerophosphatc (Sigma-Aldrich). The medium was replaced every 2 days for 14 days.

To induce chondrogenic differentiation. MSCs (passages P0-P2) were seeded at 5×10⁵ cells/drop on uncoating plastic plates to form a pelleted micromass and cultured in chondrogenic medium. Chondrogenic medium consists of low-glucose DMEM (Gibco) supplemented with 2% Fetal bovine serum (FBS, Biological Industry), 1% penicillin-streptomycin-amphotcricin (PSA, Biological Industry). 50 μg/mL L-ascorbic acid 2-phosphate (Sigma-Aldrich), 100 μg/ml, sodium pyruvate (Sigma-Aldrich), 40 μg/mL proline (Sigma-Aldrich), 10 ng/mL TGF-β2 (Invitrogen), and 50 mg/mL ITS⁺ premix (Sigma-Aldrich; 6.25 μg/mL insulin, 6.25 μg/mL transferrin, 6.25 ng/mL selenius acid, 1.25 mg/mL bovine scrum albumin, and 5.35 mg/mL linoleic acid). The medium was replaced every 2 days for 21 days.

To induce adipogenic differentiation. MSCs (passages P0-P2) were seeded at 1×10⁴ cells/cm² on tissue culture plastic plates and cultured in adipogenic medium. Adipogenic medium consists of low-glucose DMEM (Gibco) supplemented with 10% Fetal bovine scrum (FBS. Biological Industry). 1% penicillin-streptomycin-amphotericin (PSA. Biological Industry). 10 mg/ml insulin (Sigma-Aldrich), 0.2 mM indomethacin (Sigma-Aldrich), 1 mM dexamethasone (Dex, Sigma-Aldrich). 0.5 mM 3-isobutyl-1-methylxanthine (IBMX, Sigma-Aldrich). The medium was replaced every 2 days for 14 days.

1.5 Bioreactor System

The bioreactor system used in this study were described previously [30, 31]. Briefly, the bioreactor system could be divided into two parts; cell culture tank and culture medium tank. The cell culture tank of the bioreactor system was composed of a 50 mL sterile centrifuge tube and a glass casing pipe for mass transferring. The culture medium tank of the bioreactor system is a 500 mL glass bottle with a plastic cap, which comprised 4 stainless ports for medium and gas perfusion. All consumables of the bioreactor system were sterilizable via autoclaving or EO sterilization. The whale system were installed inside an incubator with humidified air (37° C. 5% CO₂). The flow rate was 1 mL/inin controlling via a peristaltic pump (LongerPump), which provided continuous medium replenishment.

1.6 Generation of Bone-Like Tissues

The alginate scaffolds were sterilized with 75% ethanol. The hMSCs were suspended in medium and then seeded into scaffolds at a density of 5×10⁵ viable cells/scaffold. The scaffolds with hMSCs (hMSCs-scaffolds) were placed in a 24-well culture plate for 24 h for cell adhesion, and then cultured in the bioreactor system containing osteogenic medium for 7, 14, 21, and 28 days. The medium was circulated with an initial pump setting of 1 mL/min via a peristaltic pump. After culture, bone cells were derived from the hMSCs, and these cells comprising the bone cells and the hMSCs were aggregated to form a cell cluster, embedding in the porous structure of the alginate scaffolds, forming hone-like tissues which were then collected and harvested via centrifugation.

1.7 Cell Proliferative Quantification

Cell proliferative quantification was assessed through Alamar Blue (Life Technologies) assay and Scepter™ 2.0 Cell Counter (Merck Millipore). Alamar Blue reduction ability of the cells in the bone-like tissues was assayed in accordance with the manufacturer's protocol. In brief, working solution was comprised of 10× dilution from stock Alamar Blue reagent with scrum-free LG-DMEM. The bone-like tissues as generated was reacted with 2 ml working solution in 15 ml sterile centrifuge tube in an incubator for 1 h and was kept in the dark. The relative fluorescence response of Alamar Blue reduction was measured at 530 nm excitation and 590 nm emission using a fluorescent microplate reader (SpectraMax M5, Molecular Devices) and present the mitochondrial activity.

Scepter™ 2.0 Cell Counter uses the Coulter principle of impedance-based particle detection. According to the manufacturer's protocol, alginate scaffolds were dissolved by EDTA solution at the beginning. The cell clusters were treated with 1× trypsin-EDTA solution to breakdown the structure into single cell. The resultant single-cell suspension was diluted to a total volume of 100 μL in phosphate buffered 1× PBS in a 1.5 mL microcentrifuge tube. Scepter™ 2.0 Cell Counter was used to detect the cell numbers directly.

1.8 Live/Dead Staining of Bone-Like Tissues Containing Alginate Scaffolds

After being cultured for 1, 7, 14, 21, and 28 days, the resultant bone-like tissues containing the scaffolds were stained with 4 μM calcein AM (ex/cm˜495 nm/˜515 nm, Life Technologies) and 4 μM of propidium iodide (PI, ex/cm˜540 nm/˜615 nm, Life Technologies) for 30 min. Live cells were stained by calcein AM, and dead cells were stained by PI. Samples were observed via a con focal microscope (LSM 780, Zeiss) and 3D images were reconstructed.

1.9 Caspase 3/7 Staining

CellEvent® Caspase-3/7 Green ReadyProbes® Reagent (Life Technologies) is a fluorogenic, no-wash indicator of activated caspase-3/7 for live- and fixed-cell applications. The bone-like tissues containing the scaffolds were reacted with CellEvent® Caspase-3/7 Green Ready Probes® Reagent for 30 min, and the counterstained with 1 μg/ml Hoechst 33342 for 5 min. The cells destined for cell death would be observed (ex/em˜502 nm/˜530 nm) by confocal microscope (LSM 780, Zeiss), and 3D cell images were reconstructed

1.10 JC-1 Staining

Fluorescent probe JC-1 (Life Technologies) was used to study the mitochondrial membrane potential (Δψm) and monitor mitochondrial health. Cells with higher mitochondrial membrane potential predominantly contain JC-1 in aggregated form, and they should show fluorescence (ex/em˜514 nm/˜590 nm); when the ΔψM Dissipates, JC-1 staining show predominantly a monomeric form emitting fluorescent (ex/cm˜514 nm/˜529 nm). The bone-like tissues containing the scaffolds were incubated with JC-1 working solution for 30 min, and the counterstained with 1 μg/ml Hoechst 33342 for 5 min. The treated bone-like tissues were washed twice with 1× PBS, then visualized via by con focal microscope (LSM 780, Zeiss), and 3D cell images were reconstructed.

1.11 MitoTracker Red FM Staining

MitoTracker® Red FM (Life Technologies) is a red-fluorescent dye that stains mitochondria in live cells and its accumulation is dependent upon membrane potential. The bone-like tissues containing the scaffolds were reacted with the MitoTracker® Red FM working solution for 45 min, and the counterstaincd with 1 μg/ml Hoechst 33342 for 5 min. The mitochondrial mass would be observed (ex/em˜581 nm/˜644 nm) by confocal microscope (LSM 780, Zeiss), and 3D cell images were reconstructed.

1.12 Xylenot Orange Staining

To perform biomineralization examination, the bone-like tissues containing the scaffolds were fixed by 4% para-formaldchydc (Asymetrix). The calcified area of bone-like tissues were reacted with 20 μM xylenol orange (Sigma-Aldrich) for 15 min, and the counterstaincd with 1 μg/ml Hoechst 33342 for 5 min. The calcified area would display in bright orange-red (ex/em˜440 nm/˜610 nm) by confocal microscope (LSM 780, Zeiss), and 3D cell images were reconstructed.

1.13 3D Micro-Computed Tomography

Before histological processing, total bone density and relative bone volume of the bone-like tissues containing the scaffolds were analyzed via a micro-CT instrument (SkyScan 1176, Bucker). Results of volumetric bone mass density (vBMD) were expressed in mg/cm³. The data were reconstructed and showed in three dimensions.

1.14 DMMB Assay

At specified time points, the release culture medium was collected and stored at −80° C. Samples were plated in triplicate with the addition of 1,9-dimethylmethylene blue (DMMB, Sigma-Aldrich) reagent. These were incubated at room temperature for 15 min and read the absorbance at 525 nm using a microplate reader (SpectraMax M5, Molecular Devices). A standard curve was generated using chondroitin 6-sulfate (C6S) substrate and samples were normalized to DNA content as assessed by PicoGreen assay (Life Technologies).

1.15 ALP Activity

The ALP activity was measured by Alkaline Phosphatase Activity Fluoromctric Assay Kit (BioVision) in accordance with the manufacturer's protocol. The data was measured at 360 nm excitation and 440 nm emission using a fluorescent microplate reader (SpectraMax M5. Molecular Devices). A standard curve for total ALP activity was generated using 4-Methylumbelliferyl phosphate disodium salt (MUP) substrate with detection sensitivity ˜1 μU.

1.16 X-Ray Diffraction (XRD)

XRD spectrum of the biological appetites were measured by Rigaku X-Ray Powder Diffractometer in 2θ ranging from 20° to 60° to find out the structure and lattice parameters of die biological appetites.

1.17 Fourier Transform Infrared Spectroscopy (FT-IR)

The biological appetites were dried at 50° C. for 48 h and then prepared as pellets with potassium bromide (KBr) powder. The chemical structures were analyzed by FT-IR spectroscope (Jasco) and the FT-IR spectra were recorded in the wave number range of 4000-400 cm⁻¹ with 62 scans per sample cycle.

1.18 Q-PCR Quantification

The bone-like tissues containing the scaffolds were dissolved in 50 mM EDTA solution at 37° C. for 5 min and the cells were collected by brief centrifugation. Total RNA was extracted from hMSCs using Total RNA Miniprep Purification Kit (GeneMark) after 1, 7, 14, 21 and 28 days' 3D culture. The total RNA were reverse-transcribed into cDNA by using Thermo Scientific First Strand cDNA Synthesis kit in accordance with the manufacturer's protocol. 5 μL of 5× OmicsGrcen qPCR Master Mix (Omics), 10 μL of primers, and 10 μL of cDNA were mixed in a final volume of 25 μL for single reaction. B2M was used as the endogenous housekeeping gene. Genes examined were inducible CD73, CD90, CD105, Alp1, Runx2, Bglap. Ostcopontin (OPN), BMP-2, vascular endothelial growth factor-A (VEGF-A), Col1a1, type 11 collagen (Col2a1), and matrix metalloprolsinase-3 (MMP-3). Reaction was performed by ABI PRISM 7500 Sequence Detection System (Life Technologies) and the PCR conditions were denaturation at 95° C. for 10 sec, annealing at 60° C. for 20 sec, and extension at 72° C. for 34 sec for up to 40 cycles. The data of relative quantitation value of gene expression was calculated using the expression of 2^{-ΔΔCt}.

1.19 Enzyme-Linked Immunosorbent Assay (ELISA)

The amount of secreted-form growth factors and bone-related protein of culture media was quantified using human ELISA kits. Genes examined were inducible Transforming growth factor beta 1 (TGF-β1, cBioscience). OCN (eBioscience), osteoprotegerin (OPG, R&D System), BMP-2 (R&D System), sCD105 (cBioscience), fibroblast growth factor (FGF, R&D System), the stromal cell-derived factor 1 (SDF-1α, also called CXCL12, R&D System), and VEGF-A (PeproTech). At specified time points the release medium was collected and stored at −80° C. The release of secreted-form growth factors arid bone-related protein was quantified up to 28 days.

1.20 Subcutaneous Implantation in NOD/SCID Mice

The procedures were performed in accordance with the guidelines for animal experimentation by the Institutional Animal Care Committee, National Taiwan University College of Medicine (IACUC No. 20130506). The bone-like tissues were fabricated with 0.3 mg/ml collagen at an initial seeding density of 1×10⁶ cells/ml. Forty-eight NOD/SCID male mice (25-30 g) were anaesthetized with 1% Isoflurane and divided into four groups (n=12). An incision was made at the back to create a subcutaneous pocket of 3×3 cm and the bone-like tissue were implanted by subcutaneous injection with G23 injection needle. Animals were observed by micro-CT after post-implantation at 1 day. 2 weeks, 1 month, and 2 month and sacrificed by CO₂ asphyxiation. The skin flaps at the implantation site were harvested for the following experiment.

1.21 Hematoxylin/cosin (h&E) and Immunohistochemical (IHC) Staining

At the end of the cultivation (day 7, 14 and 21), the resultant bone-like tissues were removed at each time-point for histological examination. Hematoxylin and cosin staining was carried out for investigating the morphology of the bone-like tissues, and immunohistochemical observation was made for the expression of type II collagen and aggrecan. Briefly, paraffin-embedded tissue block were cut into 5 μm thickness for staining. After deparaffinized and rehydrated process, endogenous peroxidases were blocked by 0.1% hydrogen peroxide (Sigma-Alderich, USA) in PBS solution for 10 min. For retrieval process, nonspecific background staining was blocked by 20 μg/mL proteinase K (Sigma-Alderich. USA) solution and incubated 20 min at 37° C. in humidified chamber. Primary antibodies, rabbit anti-type II collagen (Abeam. USA) and rabbit anti-aggrecan (GeneTex, Taiwan), were added with appropriate dilution on the tissue sections and incubated at 4° C. overnight. After incubation, rinse tissue sections and then incubate with SuperPicture™ Polymer Detection Kit (Life Technologies, USA) for 10 min at room temperature. Finally, the tissue sections were revealed by 3.3′-diaminobenzidine (DAB, Sigma-Alderich, USA) substrate solution. For all the tissue section staining protocols, hematoxylin was used as counterstain of the slides.

1.22 Statistical Analysis

Statistical analysis was conducted at least in triplicate, and all the results were presented as the mean±standard deviation (SD). Statistical analysis was performed for all the quantitative results using Student's t-test for comparing means from two independent sample groups. A difference of p values less than 0.05 was considered statistically significant.

2. Results

2.1 Culturing MSC-Alginate Scaffolds in a Perfused Bioreactor System for Micro-Tissue Formation

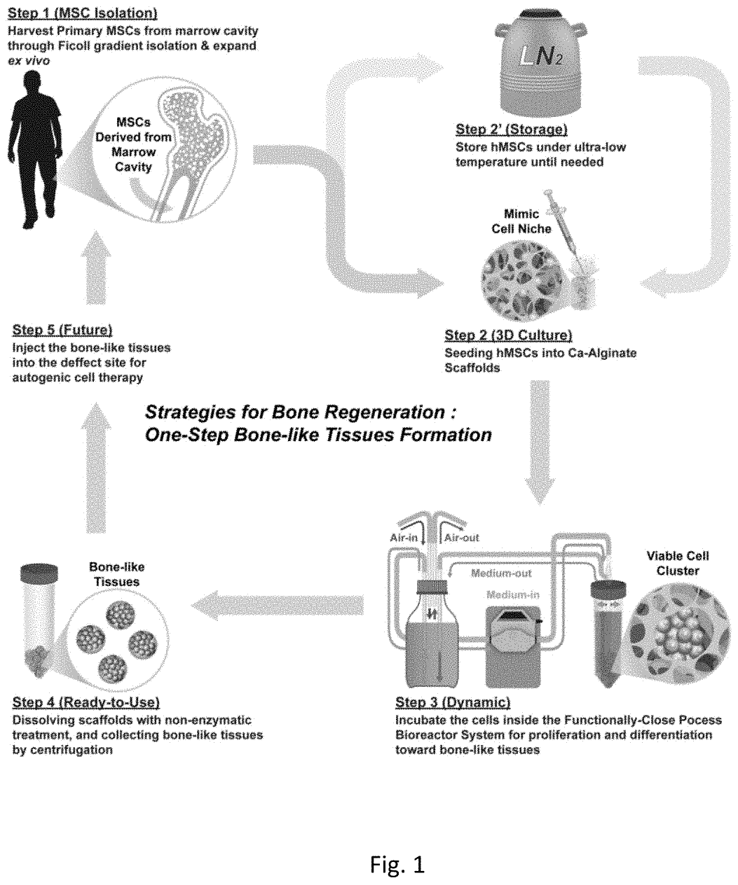

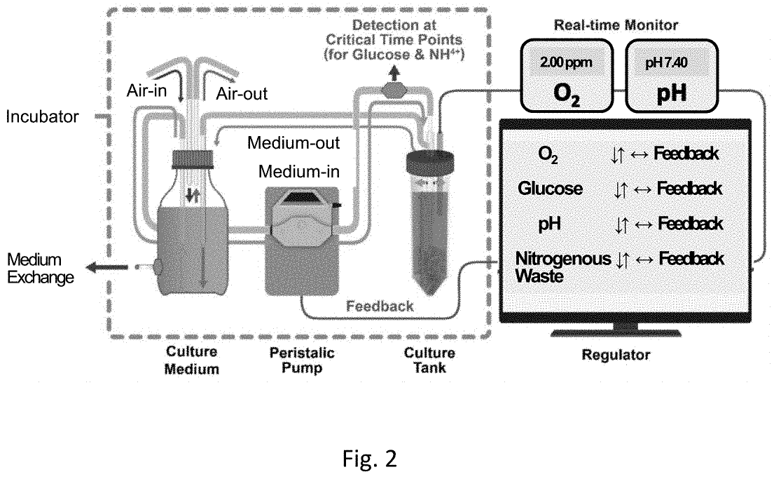

In this study, a MSC microenvironment using a perfused bioreactor system has been created for micro-tissue formation as a model to create a 3D tissue-like cell cluster via one-step rule (after MSCs adhering to an alginate scaffold, the resultant MSCs-alginate construct can be directly transferred to a perfusion bioreactor system such that both cell differentiation and proliferation are carried out in die scaffold and a 3D tissue-like cell cluster comprising MSCs and specific cells of interest differentiated from the MSCs is produced that is useful for tissue transplantation (FIG. 1); while conventionally it is required to either obtain differentiated (primary) cells from an individual or culture MSCs in a 2D condition for expansion and differentiation first for preparing an implant priori to transplantation). Specifically, at the beginning, cells were harvested from bone marrow cavity at surgery, hMSCs were purified using Ficoll-Plaque PLUS solution, and expanded ex vivo to obtain sufficient amounts of cells (Step 1). The isolated hMSCs were stored under ultra-low temperature for further use: conversely, the cells were seeded into alginate scaffolds for 3D culture directly (Step T and Step 2). Alginate scaffolds provide highly porous structures and offer a relative soft growth environment as cell niche. After seeding into the scaffolds, the hMSCs—Alginate constructs were transferred into a perfused bioreactor system in osteogenic medium containing dexamethasone, ascorbic acid 2-phosphate, beta-glycerophosphatc, and FBS (20%) for cultivation for 7, 14, 21, and 28 days (Step 3). After the specified periods of incubation, bone cells were derived from the hMSCs and these cells comprising the bone cells and the hMSCs were aggregated to form a cell cluster, embedding in the porous structure of the alginate scaffolds, forming bone-like tissues. The resultant bone-like tissues can be further treated with a chelating agent e.g. EDTA to dissolve the scaffolds, so as to generate scaffold-free bone-like tissue without enzymatic treatment. The bone-like tissue was harvested by simple centrifugation (Step 4). The bone-like tissue is injectable and has potential to be applied on autologous bone transplantation (Step 5).

2.1.1 Identification and differentiation of hMSCs

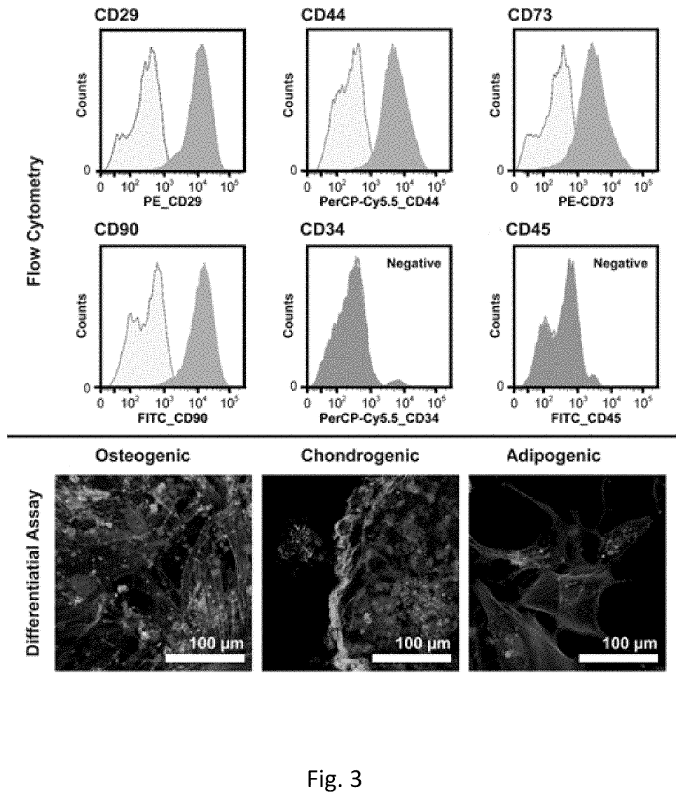

The expression of specific cell surface markers CD29, CD44, CD 73, CD90, and hematopoietic CD34 and CD45 were analyzed by flow cytometry. Fluorescent cell screening of undifferentiated hMSCs, as shown in FIG. 3, CD29, CD44. CD73 and CD90 presented positive signals; on the contrary, the expression of CD34 and CD45 were negative. Through the flow cytometric data, we demonstrated the cells we harvested preserved sternness.

FIG. 3 showed the differential capability of hMSCs. In FIG. 3 (lower parts, left), hMSCs differentiated into osteo-like cells in 14 days. FIG. 3 (lower parts, middle) revealed that hMSCs differentiated into chondro-like cells in 21 days via pellet culture. FIG. 3 (lower parts, right) presented that hMSCs differentiated into adipo-like cells in 14 days. The F-actin molecules and the nucleus was also stained and observed. According to the data, these hMSC's can be utilized for the following experiments.

2.1.2 Live/Dead Staining of hMSC Cell Clusters in Alginate Scaffolds

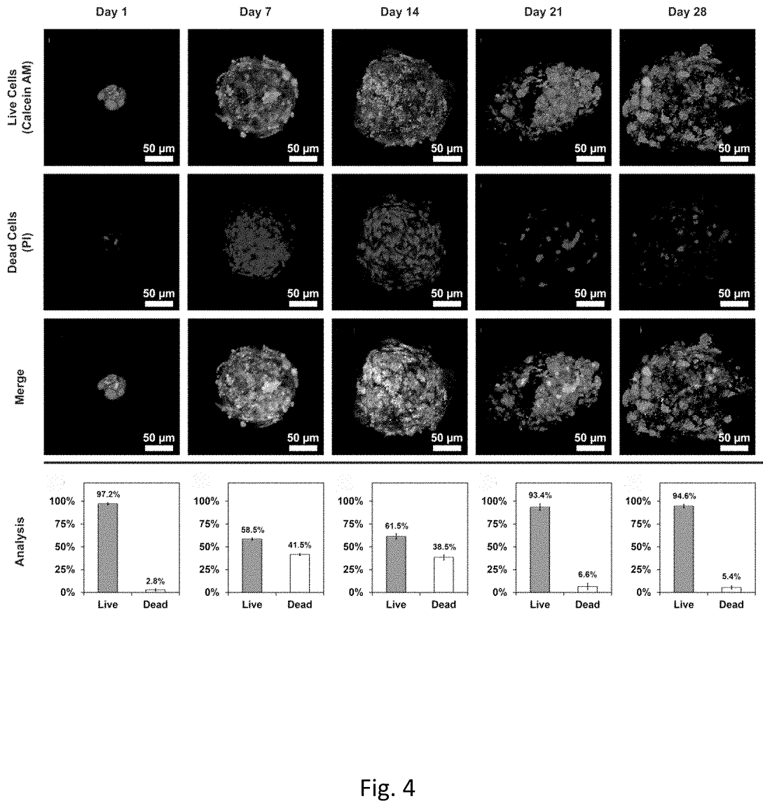

Cell viability of bone-like tissues containing the alginate scaffolds was evaluated by fluorescent staining (Calcein AM/PI) and presented in FIG. 4. At day 1. hMSCs self-assembled into cell clusters and survived in the alginate scaffolds; in sharp contrast, there were 41.5% at day 7 and 38.5% at day 14 cell death under osteogenic induction. However, only 6.6% of total cells were dead inside cell clusters at day 21 and 5.4% of total cells was found dead at day 28. Cell death was concentrated at the center of cell clusters, and there were some vacancies occurred inside the hMSC cell clusters. In the past, scientists had already proven that hMSCs would increase the sensitivity to apoptosis during differentiation, even at the very early stages [32]. Consequently, live alginate scaffolds in the perfusion bioreactor system created an environment permissive for hMSCs differentiation and cell clusters formation.

2.1.3 Apoptotic and Mitochondrial Transmembrane Potential Detection of hMSC Cell Clusters

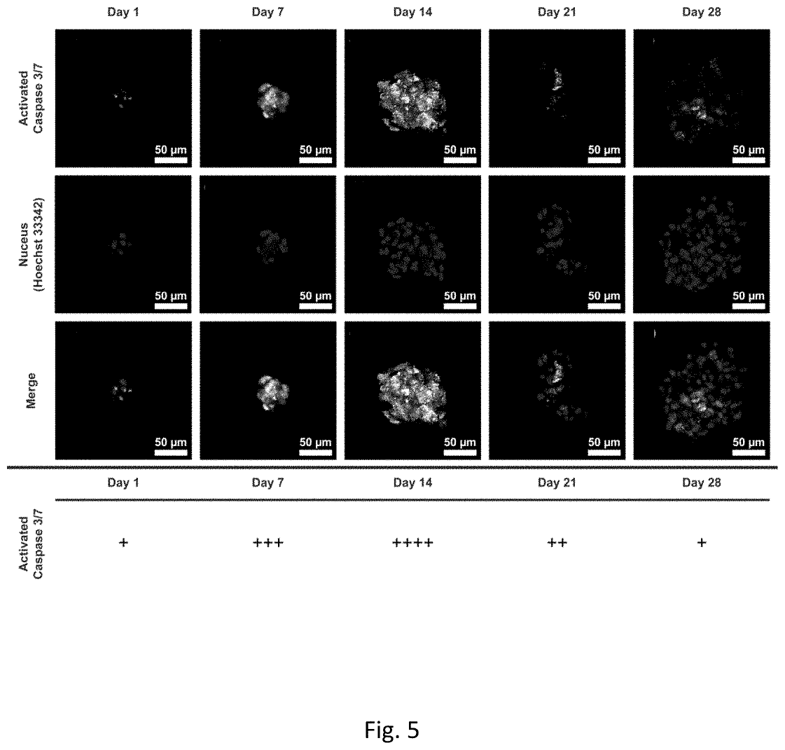

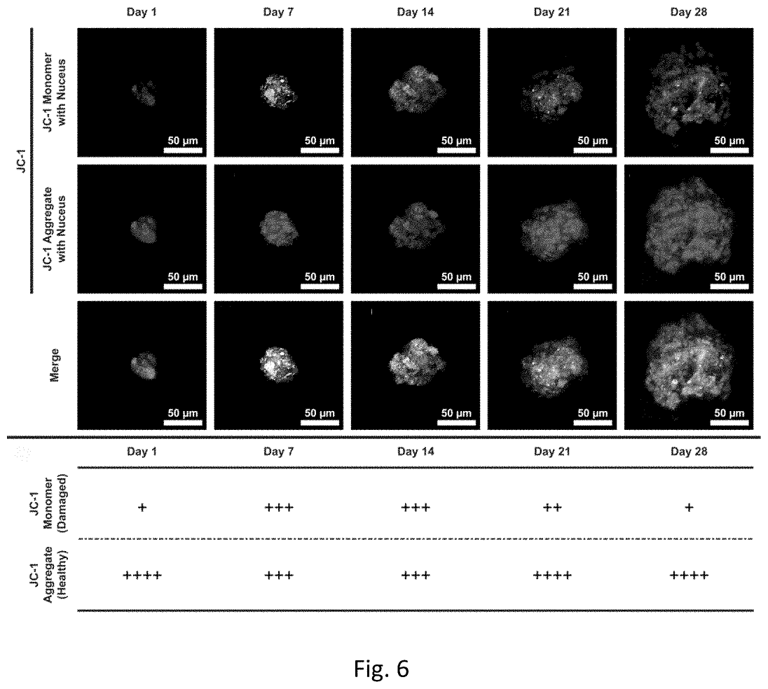

The results of Live/Dead staining suggested that a dramatic cell death of cell clusters was accompanied with differentiation, so we investigated caspase 3/7 activity for apoptotic detection (FIG. 5) and checked cell health via mitochondrial transmembrane potential examination (FIG. 6). The activation of apoptotic caspascs 3/7 significantly increased at day 7 and day 14, corresponding to Live/Dead staining data, the cell death might be caused by caspase-mediated apoptosis. The mitochondrial transmembrane potential examination represented the same tendency. Therefore, we suggested the cell clusters were toward differentiation and accompanied activation of apoptosis in the bioreactor system.

2.1.4 Mitochondrial Mass and Morphology of hMSC Cell Clusters

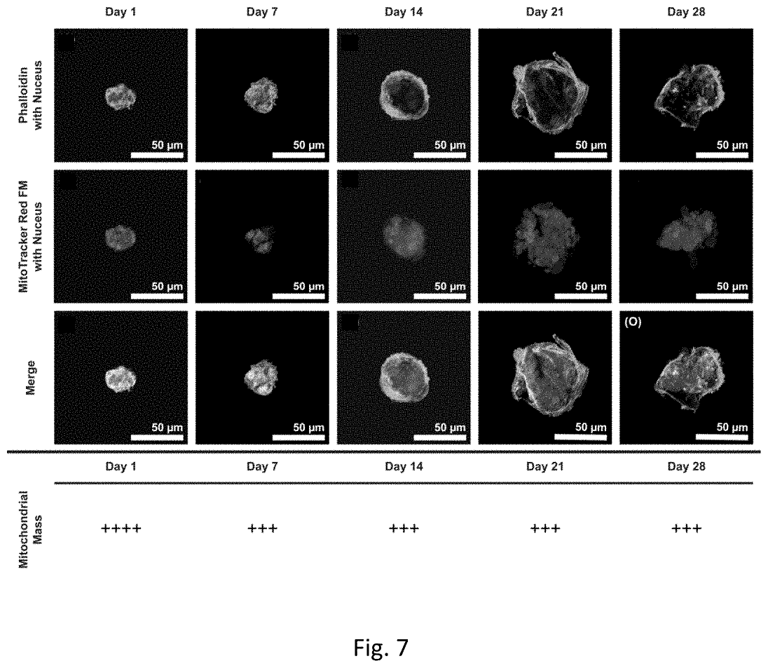

The structure of bone-like tissues was assessed using phalloidin labeling, and mitochondrial mass was determined using MitoTracker Red FM (FIG. 7). In FIG. 7, phalloidin conjugated with fluorescent signal and showed the structure of hMSC cell clusters. Additionally, MitoTracker Red FM presented the mitochondrial mass slightly decreased during cultivation in the bioreactor system ( from day 1 to day 28, left to right).

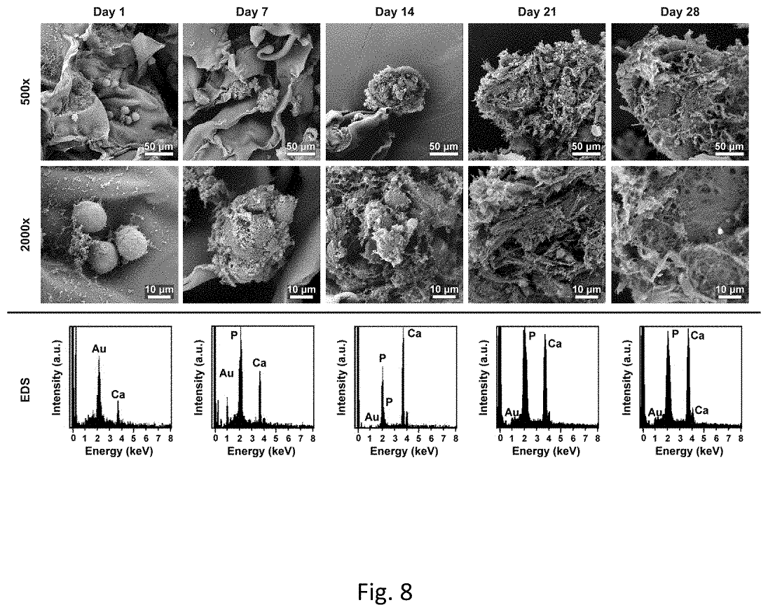

The morphology of hMSCs in the alginate scaffolds was observed by SEM, and die calcium/phosphorous signals were evaluated by SFM with FDX. At day 1, individual cells distributed in a random pattern within the alginate scaffolds, and only calcium signal from scaffolds were detected through the EDX measurement (FIG. 8, lower parts, day 1). Under dynamic perfusion. hMSCs aggregated into cell clusters surrounding with abundant ECM (FIG. 8, lower parts, from day 7 to day 14). Moving on to the EDX examination, die data indicated that there were biological apatite organized at the surface of the hMSC cell clusters as time goes by (FIG. 8, lower parts, from day 7 to day 28). These hMSC cell clusters presented 3D structures and exhibited biomineralization, suggesting that the alginate scaffolds integrated with the perfusion bioreactor system supply a suitable environment for MSCs for bone-like tissue formation.

2.1.5 Evaluation of Endochondral Ossification

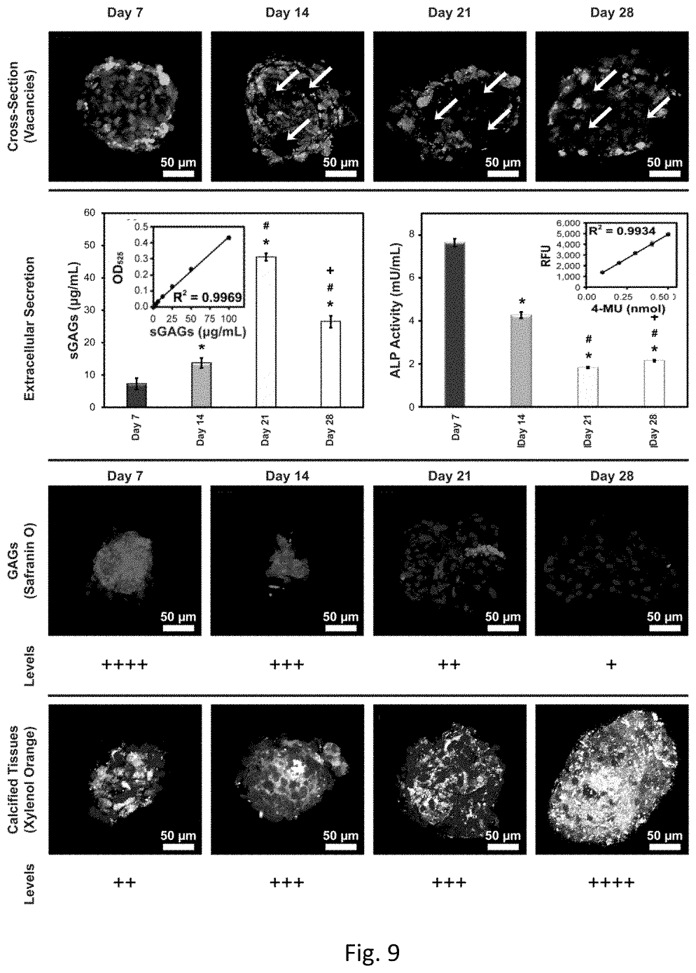

Following to the data in FIG. 9, the cross-section view of Live/Dead staining was showed at the first row (FIG. 9, upper parts, first row, the cross-section images), where the white arrow indicated there were some vacancies occurring inside the bone-like tissues. We hypothesized these vacancies might be composed of ECM and calcified tissues, so we checked the extracellular secreted glycosaminoglycan (sGAG) levels and ALP activity. The sGAG serves as cartilage-specific proteoglycan and the releasing form in culture media was exanimated by DMMB quantitative method, and the data showed that sGAG level decreased after Day 21 (FIG. 9, upper parts, second row, left). On the other hand. ALP is an early osteogenic marker and the activity was decreased over time (FIG. 9, upper parts, second row, right). According to the data revealed, the osteogenesis began during the first 7 days and was accompanied by chondrogenic differentiation. For that reason, we suggested the bone-like tissues got toward mature bone tissues via endochondral ossification.

2.1.6 Biomineralization of Bone-Like Tissues

The process of biomineralization is forming organic-inorganic hybrid composites via biological production in bone formation. XO is a fluorochrome widely used for labeling calcified tissues. Following a specific period of incubation, the calcified area of bone-like tissues was examined with XO (FIG. 9, lower parts). According to the data of the cross-section view (FIG. 9, upper parts) and XO staining (FIG. 9, lower parts), we suggested the calcified tissues replenished the vacancies inside the bone-like tissues and got toward mature tissues as time goes by. These results demonstrated that the alginate scaffolds combining with inductive osteogenic supplements can provide a suitable environment for biological minerals production and regulate bone maturation.

2.1.7 Volumetric Bone Mineral Density (vBMD) and Bone Volume

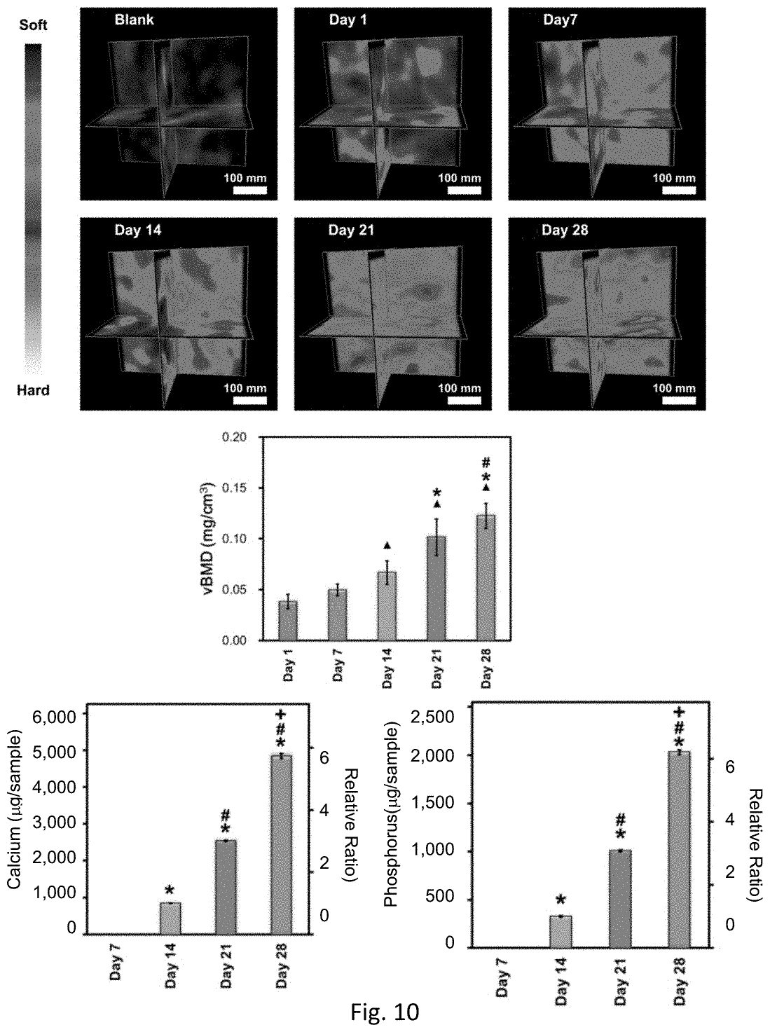

3D reconstructions were obtained by stacking 2D images, and 3 regions of interest were chosen randomly from the full view of the alginate scaffolds for analysis by micro-CT with a 9-μm isotropic voxel size resolution. The data represented that scaffolds were getting harder through the time (FIG. 10, upper parts). The vBMD value was quantified by using the algorithm provided in the supplied software (CTAn 1.14, Bucker, Belgium). After 7, 14, 21 and 28 days' perfusion, vBMD was increased over time (FIG. 10, lower parts) and indicated that the bone-like tissues obtained in this study have the potential to be applied on therapeutic treatments of bone tissue engineering.

Moreover, the amounts of calcium and phosphorus atomic elements were determined by ICP-OES. The data presented that calcium and phosphorous ions increased over time (FIG. 10, lower parts) from the biological apatite remains, which were collected in the culture media. In addition, the Ca/P atomic ratio of the biological apatite remains in the four experimental group was around 1.85-1.98. The mean values of Ca/P atomic ratio of published data were within a very wide range [33]; tracking through an element scale, the nanocrystals of biological apatite containing a variety of substitutions or vacancies, therefore, the Ca/P atomic ratio calculated in this study deviated from the chemically synthesized HAP ratio of 1.67.

2.1.8 XRD and FT-IR Determination

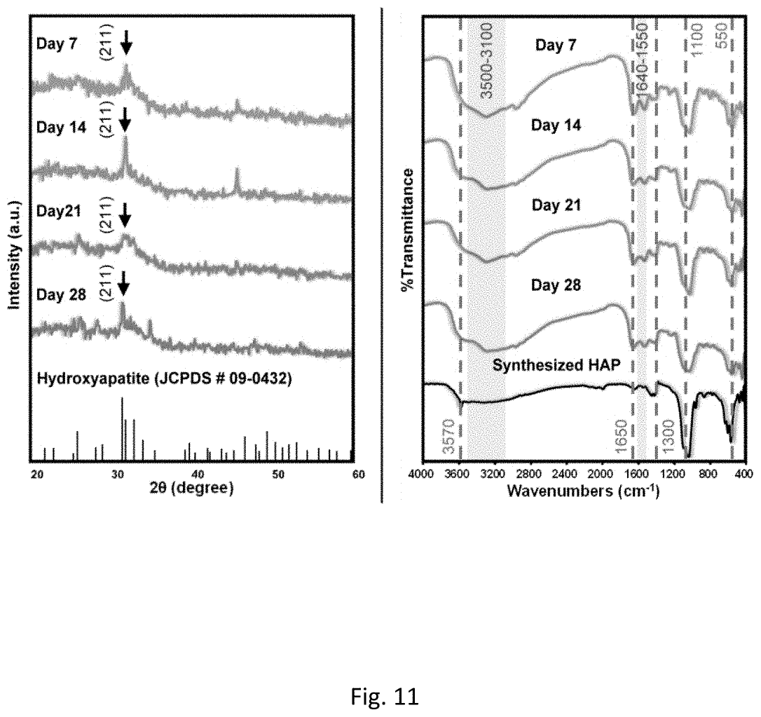

In the bodies of mammals, all normal biological mineralization and calcification consist of non-stoichiometric and ion-substituted calcium orthophosphates. In FIG. 11, left, there were specific peaks in all the groups to a specific diffraction pattern of HAP at (211) plane. Among all ion substitution, the presence of 0.5-1.5% Mg²⁺ and 4-8% carbonates (CO₃²⁻) instead of orthophosphate anions (H₂PO₄⁻ or HPO₄²⁻) is crucial particularly, because it significantly increases the solubility and leads to large lattice strain with a lower crystallinity [34].

Moving on to the analysis from FT-IR spectroscopy (FIG. 11, right), the most impressive peaks was attributed to phosphate groups, which lied at 1200-900 and 600-500 cm⁻¹. The bands of carbonate peaks displayed between 1650 and 1300 cm⁻¹, and an obvious hydroxyl bending mode was exhibited around 3570 cm⁻¹. Moreover, the broadness of primary and secondary amino groups were shown in the range of 3500-3100 cm⁻¹ and 1640-1550 cm⁻¹, which were provided by collagen or some other proteins. In accordance with the data of biomineralization, such as EDX, micro-CT, ICP-OES, XRD, and FT-IR, we had already approved that hMSCs would produce amounts of biological apatite in the bioreactor system under osteogenesis. All the evidences demonstrated the bioreactor system not only provides a suitable environment for osteogenic differentiation, but also supports the bone-like tissues toward mature bone.

2.1.9 mRNA Expression Levels of Bone-Like Tissues

To determine the relative mRNA expression levels in 3D cultivation, the data was measured by Q-PCR. In this section, we discussed gene expression and separated into four parts with different cell performance: MSC-associated surface markers, early osteogenic markers, bone-associated markers and growth factors, and ECM-related markers. The values of target gene expression were compared with Ctrl, and ail data for gene expression was normalized by Ctrl (monolayer hMSCs cultured without osteogenic induction) and calculated using the expression of 2^{-ΔΔCt}.

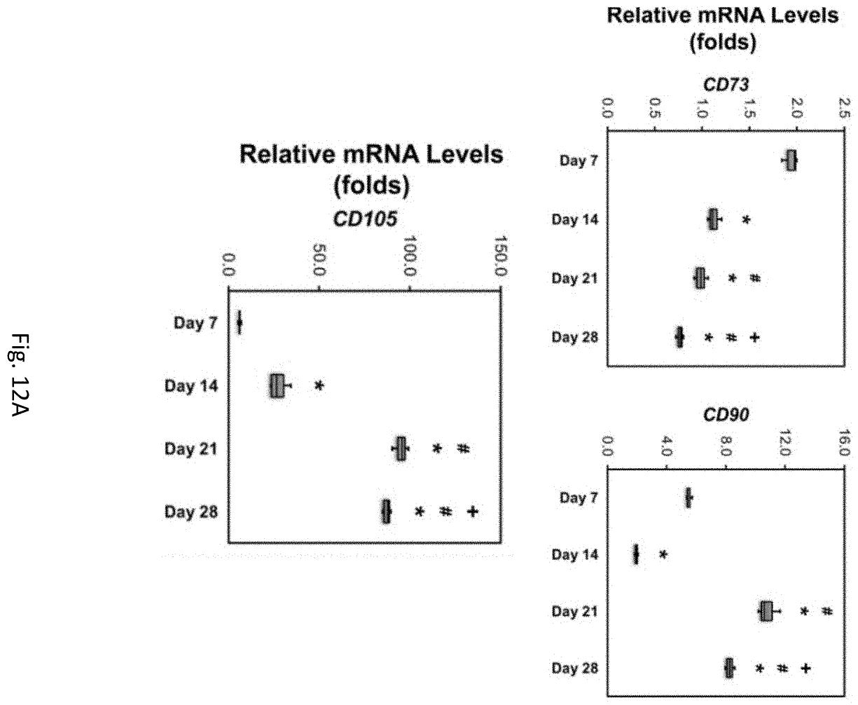

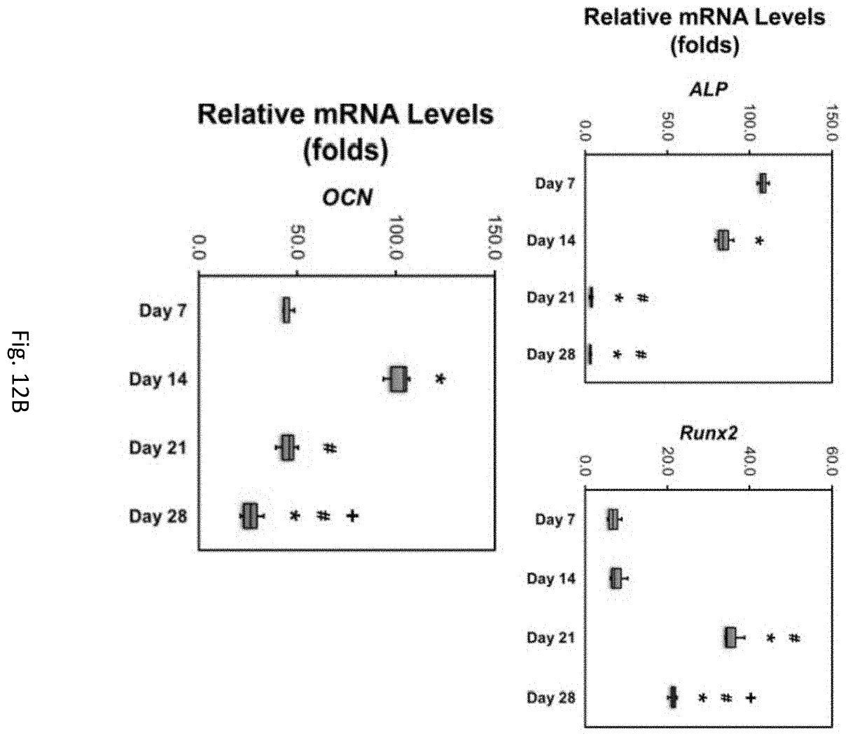

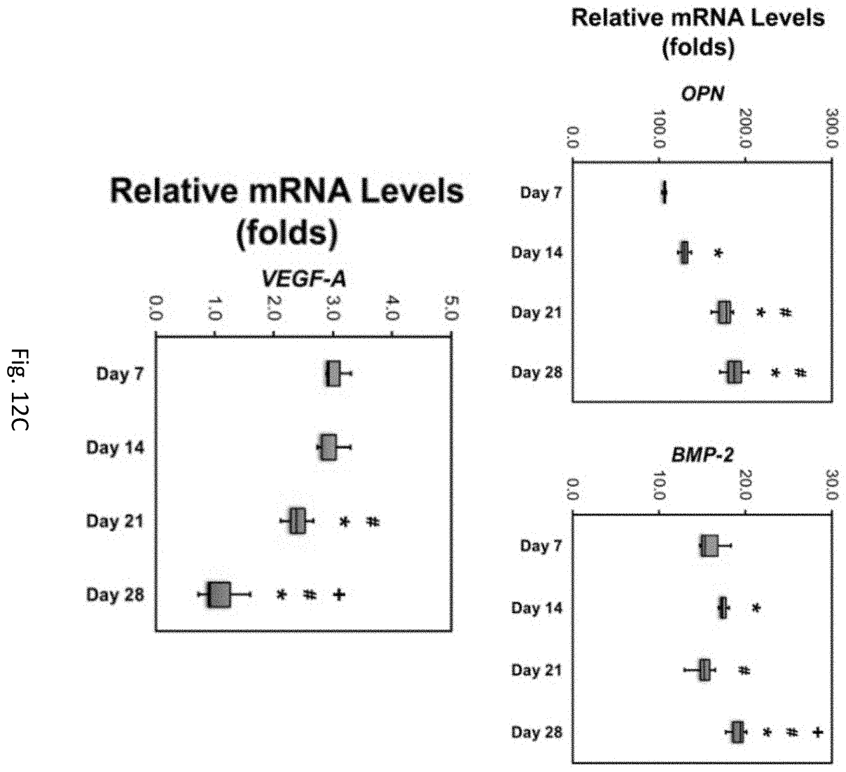

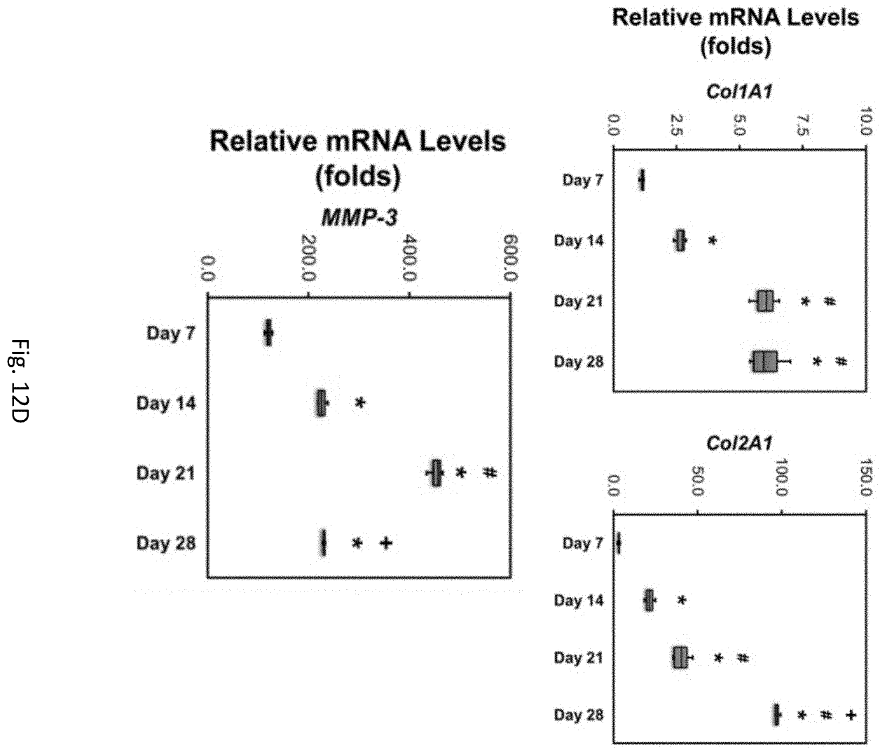

For MSC-associated surface markers, MSCs must express CD73, CD90, and CD105; following to FIG. 12A (MSC surface markers). hMSCs under osteogenic induction can upregulate CD surface marker expression in the bioreactor system at Day 7. It is totally distinct from the data revealed in 2D groups.

We also checked three early osteogenic markers; ALP encodes for a hydrolase enzyme highly expressed in bone that increased during early bone formation (FIG. 12B, early osteogenic markers); Runx2 encodes for a transcription factor required for osteogenic differentiation (FIG. 12B, early osteogenic markers); Moreover. OCN is a secreted molecule that acts as a hormone to stimulates bone formation in early osteogenic differentiation (FIG. 12C, early osteogenic markers).

Moving on to the bone-associated markers and growth factors, OPN has ability to induce undifferentiated hMSCs for the enhancement of subsequent osteogenesis, and the gene expression of OPG in this system increased over time (FIG. 12C, bone-associate marker and growth factors). During osteogenesis. BMP-2 commits to the osteogenic lineage and the mRNA levels were raised up in this system (FIG. 12C, bone-associate marker and growth factors). In FIG. 12C, VEGF-A showed an increment at the beginning, but decreased the mRNA levels as time goes by (FIG. 12C, bone-associate marker and growth factors).

ECM dictate cell behavior via instructive signals production, thus, we examined the ECM-related markers, which regulated hMSC osteogenesis. Col1a1 encodes for a major structural component of the bone ECM and the gene expression was improved over time (FIG. 12D, FCM-related gene); moreover, Col2a1 plays a primary extracellular composition of die cartilage ECM and the gene expression represented the same tendency (FIG. 12D, ECM-related gene). Besides, MMP-3 is a matrix metalloproteinase to degrade type II collagen, and it also showed the same trend (FIG. 12D, ECM-related gene). In accordance to the Q-PCR data of Col2a1 and MMP-3, we suggested hMSCs differentiated into bone-like tissues via endochondral ossification in the bioreactor system.

2.1.10 Growth Factor and Bone-Related Protein Expression Levels of Bone-Like Tissues

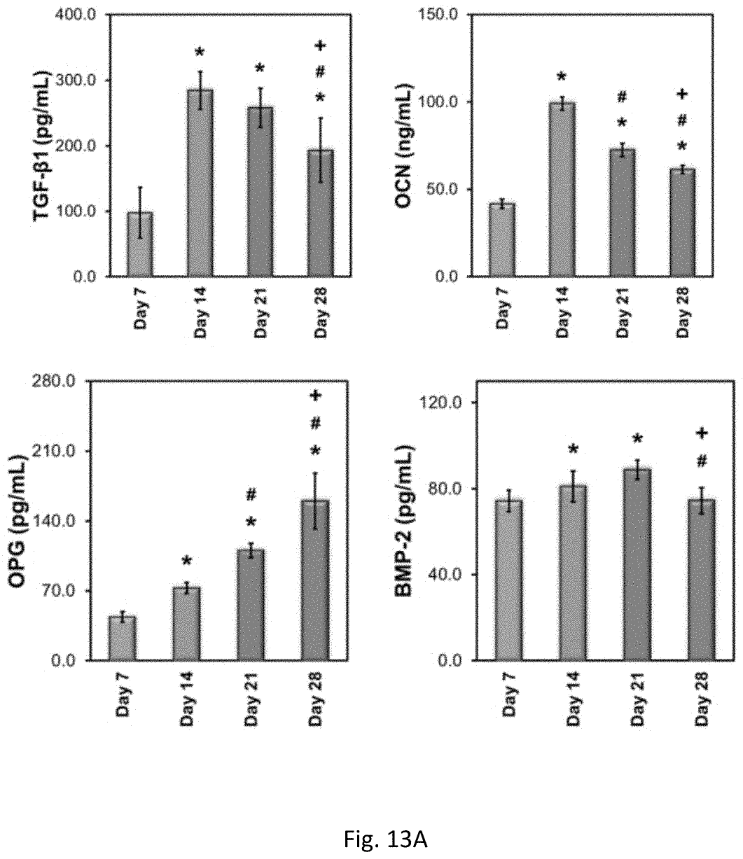

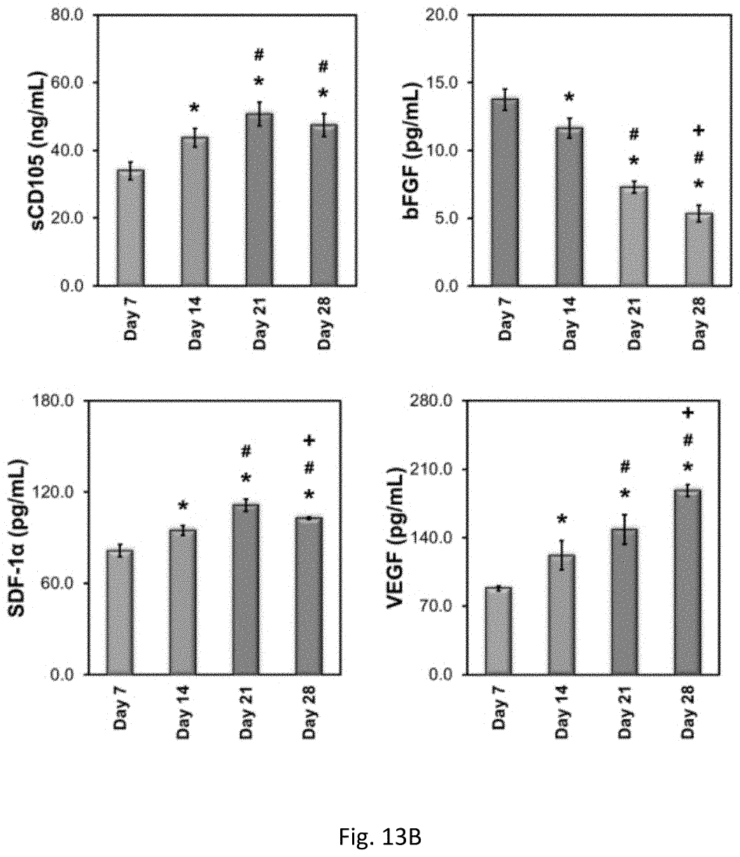

Endochondral ossification is an essential process during fetal development of the mammalian skeletal system by the replacement of a cartilage model by bone. First, we checked osteogenic-associated markers, such as TGF-β1, OCN, OPG, and BMP-2 (FIG. 13A, osteogenic markers). TGF-β1 is a key requirement to promote early chondrogenesis, and the data showed that TGF-β1 protein level decreased after Day 14 (FIG. 13A, osteogenic markers). As mentioned in last section, OCN is commonly used as an early osteogenic marker and its protein level decreased after Day 14 (FIG. 13A, osteogenic markers). OPG has ability to induce undifferentiated hMSCs for the enhancement of subsequent osteogenesis, and the amounts of OPG in this system increased as time goes by (FIG. 13A, osteogenic markers). In osteogenic differentiation, BMP-2 commits to the osteogenic lineage and were detectable in this system (FIG. 13A, osteogenic markers).

Additionally, we examined osteogenic-associated cofactors and growth factors, including sCD105, bFGF, SDF-1α, and VEGF-A (FIG. 13B. osteogenic markers). sCD105 is a soluble form of CD105 and exhibits distinct cell function for facilitating TCF-β1 signaling pathway toward osteogenic differentiation (FIG. 13B, osteogenic markers). bFGF is one of the most common growth factors and cooperatively supports sternness; the data represented that bFGF protein level decreased after Day 7 and corresponded to the gene expression of MSC-associated CD markers, which consists of CD73, CD90, and CD 105 (FIG. 13B, osteogenic markers). SDF-1α controls cell proliferation and section of VEGF, and the SDF-1α levels of secretion increased over time (FIG. 13B, osteogenic markers). Since SDF-1α might stimulate VEGF secretion, VEGF-A was discovered in this system and accumulated through the time (FIG. 13B, osteogenic markers).

2.2 Xeno-Free System

A bone-like tissue was obtained by seeding MSCs in alginate scaffolds and culturing the alginate scaffolds with MSCs in a three-dimensional, perfusion condition, as descried in Example 2.1, however, the culture medium did not include scrum and instead include xenogeneic-free/scrum substitutes e.g. UltraGRO (0.1%-10%, particularly 1%-8%, more particularly 3%-6%).

2.2.1 Live/Dead Staining of Bone-Like Tissues Xeno-Free Perfusion Bioreactor System

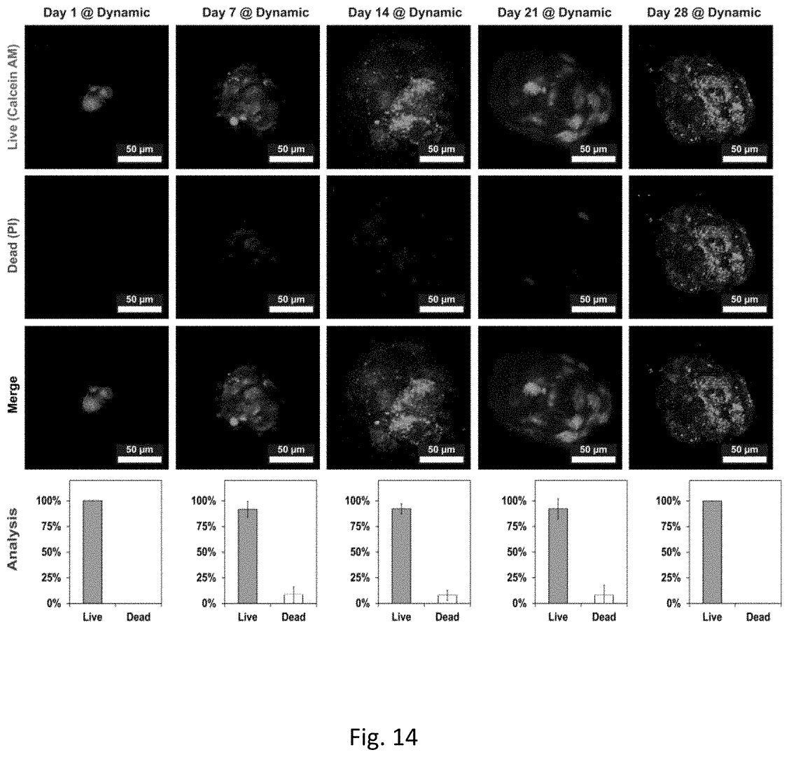

Cell viability of bone-like tissues containing the alginate scaffolds in xeno-free perfusion bioreactor system was evaluated by fluorescent stained (Calcein AM/PI) and presented in FIG. 14. At day 1. hMSCs self-assembled into cell clusters and survived in the alginate scaffolds; in sharp contrast with FBS-based system, the bone-like tissues represented excellent cell viabilities in all time periods. In the past, scientists had already proven that hMSCs under stress would increase the sensitivity to apoptosis during differentiation [35]; therefore, the data demonstrated that xeno-free supplement may provide hMSCs a stable environment and protect hMSCs from apoptotic program.

2.2.2 Live/Dead Staining of Bone-Like Tissues Under Static Condition

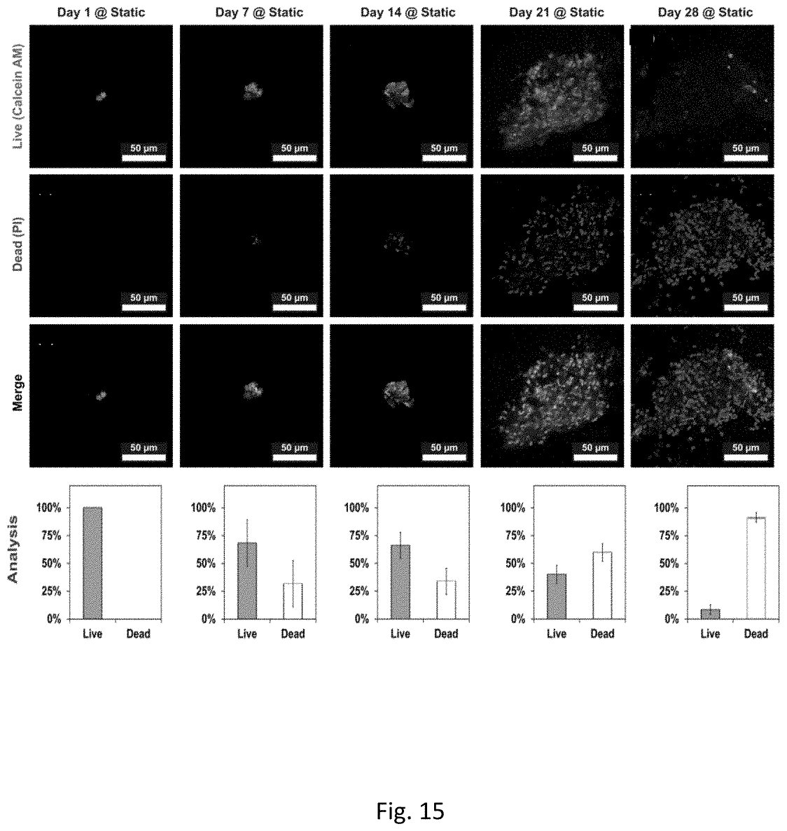

Comparing to the dynamic group of the bone-like tissues in last section, the static approach in the xeno-free system also examined with Live/Dead method. On the basis of our previous study [36], static conditions might cause obvious death of cells in 3D environment (FIG. 15), whereas hMSCs survived in the alginate scaffolds with dynamic fluids (the perfusion condition) (FIG. 14). In the static group. 31.8% of total cells were dead inside cell clusters at day 7, 33.8% of total cells were dead inside cell clusters at day 14. 59.8% of total cells were dead inside cell clusters at day 14, and 91.6% of total cells was dead at day 28. Consequently, the perfusion system played a pivotal role in maintaining cell viability.

2.2.3 Biomineralization of Bone-Like Tissues

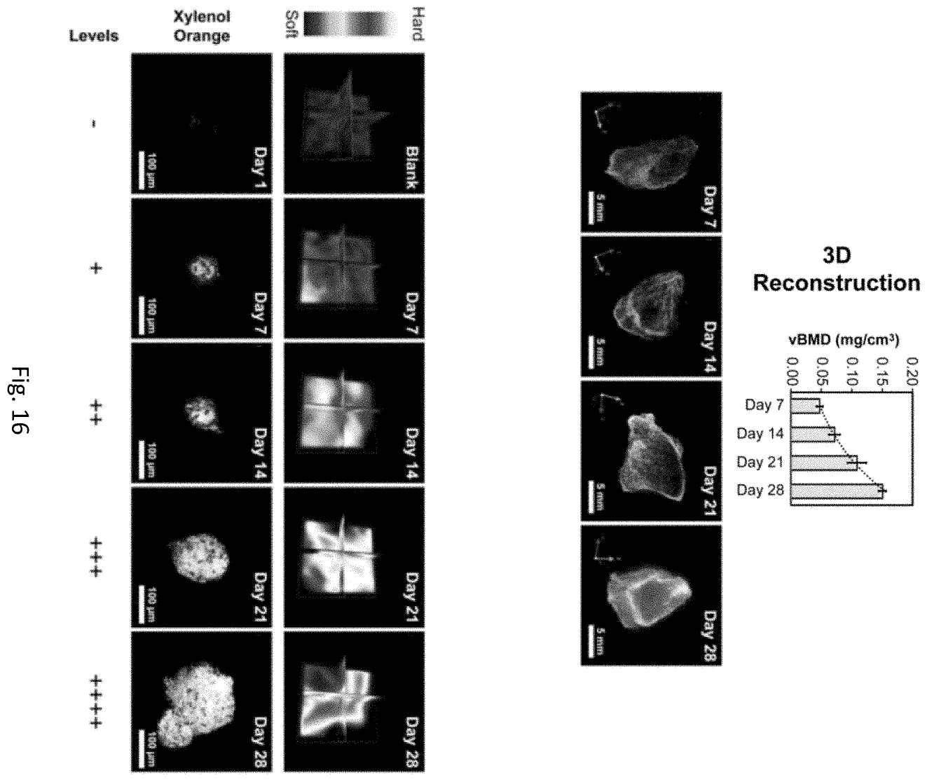

The process of biomineralization is forming organic-inorganic hybrid composites via biological production in bone formation. In this section we utilized micro-CT and XO staining method to examine biomineralization. The vBMD value was quantified by using the algorithm provided in the supplied software (CTAn 1.14, Broker, Belgium,). After 7, 14, 21 and 28 days' perfusion, vBMD was increased over time (FIG. 16, upper parts) and indicated that the bone-like tissues in xeno-free system obtained in this study also presented osteogenic activities and had potential to be applied on therapeutic treatments. 3D reconstructions were obtained by stacking 2D images, and 3 regions of interest were chosen randomly from the full view of the alginate scaffolds for analysis by micro-CT with a 9-μm isotropic voxel size resolution (FIG. 16, lower parts, first row). The data represented that scaffolds were getting harder through the time (FIG. 16, lower parts, first row, blank and day 7 to day 28).

Xylenol orange (XO) is a fluorochrome specific for calcified tissues. After the specified periods of perfusion, the calcified area of bone-like tissues was stained with XO (FIG. 16, lower parts, second row, day 1 to day 28). These results demonstrated that the alginate scaffolds combining with inductive osteogenic supplements can provide a suitable environment for biological minerals production and regulate bone maturation.

2.2.4 Volumetric Bone Mass Density for In-Vivo NOD-SCID Model

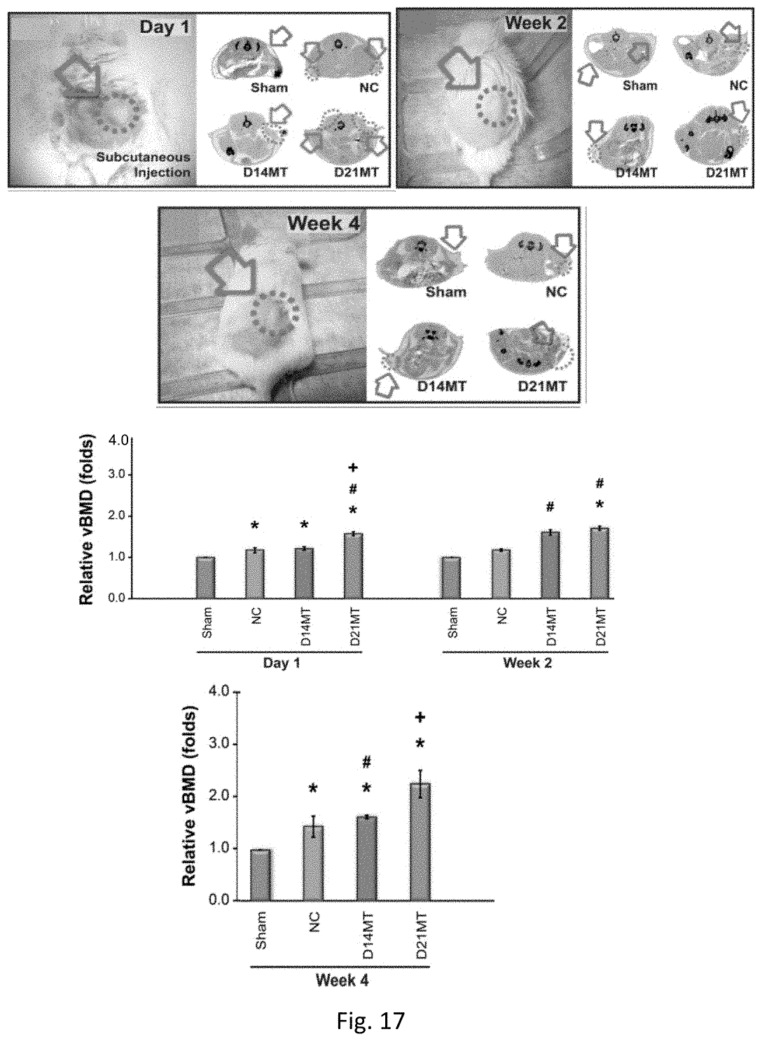

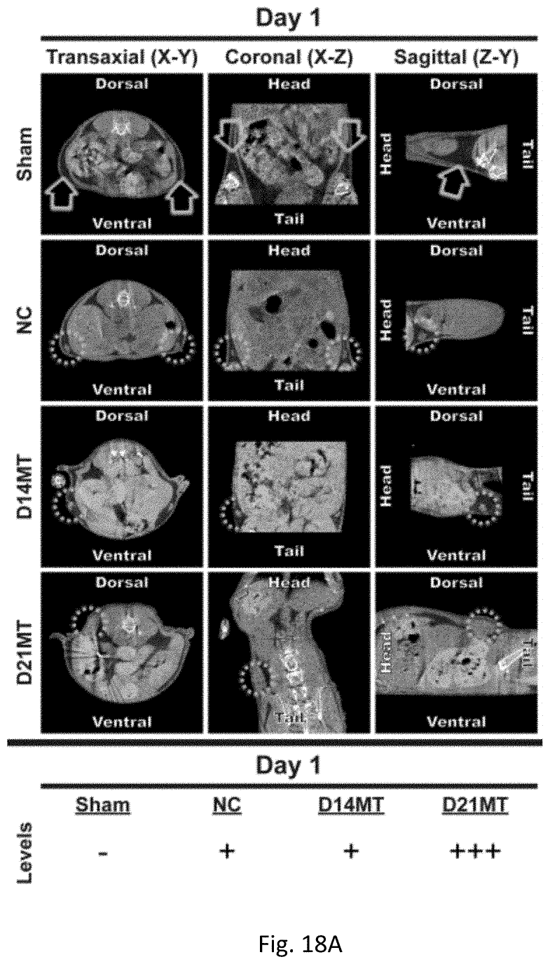

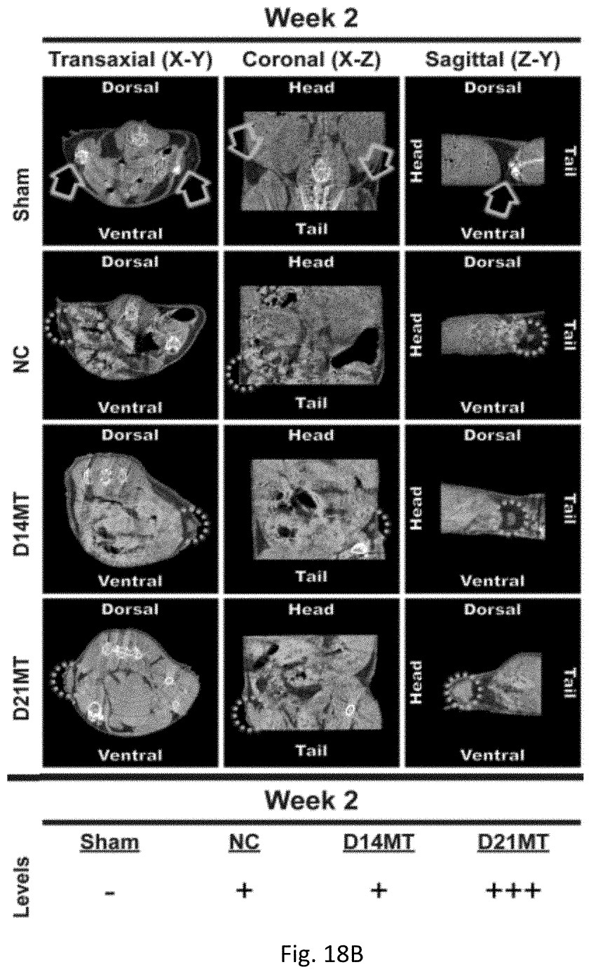

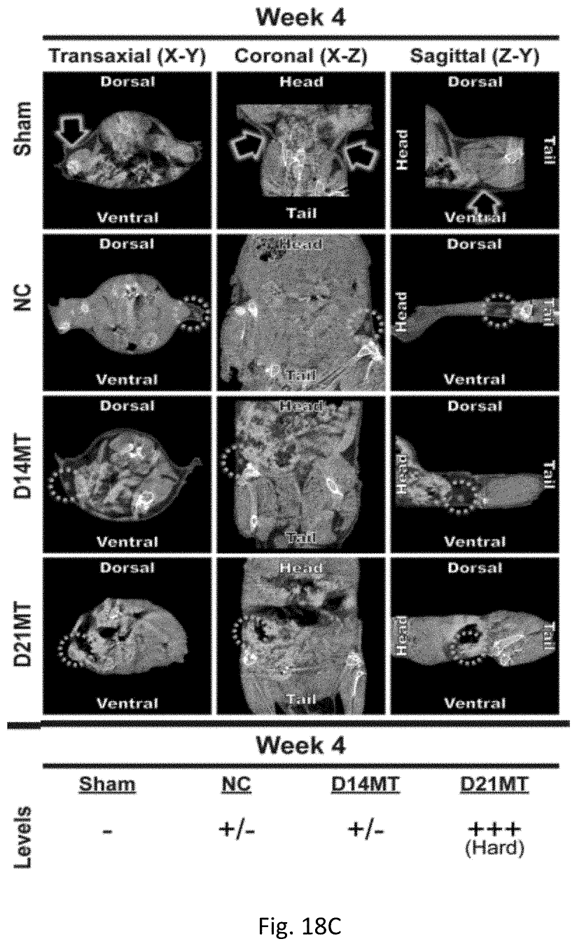

For the living NOD/SCID mice model approach, the process of the subcutaneous bone-like tissues injection was evaluated by micro-CT at a 9-μm isotropic voxel size resolution in the living NOD/SCID mice at day 1, week 2 and week 4 (FIG. 17, upper parts). The 2D images was transformed by the supplied software (DataViewer 1.5. Bucker. Belgium) and represented in FIG. 18A-18. The vBMD was calculated as percentages (%) using the algorithm provided in the supplied software (CTvox 2.4, Bruker, Belgium) and 3 regions of interest were chosen randomly from the full view of engrafted-tissues for analysis (FIG. 17, lower parts). In accordance of the data in this study, the bone-like tissues engrafted subcutaneously into NOD/SCID mice demonstrated that bone-like tissues from xeno-free system have the potential to be used in therapeutic applications.

3. Conclusions

In this study, we had developed and established a platform to generate a 3D tissue-like implant by seeding MSCs in alginate scaffolds and culturing the MSC-alginate scaffolds in a perfusion bioreactor system. For the purpose on cell therapy, mimic cell niche in vivo is a key mediator of maintaining cell capability. In the platform, hMSCs under osteogenesis, for example, can differentiate and grow into functional bone-like tissues with biomineralized structure and abundant ECM. Through the osteogenesis process in the perfusion bioreactor system as described herein, hMSCs could grow, differentiate, and assemble bone-like tissues. We had already established standard operation procedure for bone-like tissue formation and collection. These strategies could reduce the surgical procedure and form enough 3D tissue-like implant for cell therapy. This strategy could make up enough 3D tissue-like implant for cell therapy and avoid the side effects from allograft or xenograft. Overall, this study demonstrates that our system could provide a safe and affordable tool for tissue engineering.

### REFERENCES

1. Linger, R. and J. Vacanti, Tissue engineering. Science, 1993, 260(5110): p. 920-926.
2. Walgenbach, K.-J., et al., Tissue engineering in plastic reconstructive surgery. The Anatomical Record, 2001, 263(4): p. 372-378.
3. Kim, K. and G. Evans, Tissue engineering: the future of stem cells. Topics in tissue engineering, 2005, 2: p. 1-21.
4. Griffith, L. G. and G. Naughton, Tissue Engineering—Current Challenges and Expanding Opportunities. Science. 2002, 295(5557): p. 1009-1014.
5. Purnell, B., New Release: The Complete Guide to Organ Repair. Science, 2008, 322(5907): p. 1489.
6. Ikeda, E., et al., Fully functional bioengineered tooth replacement as an organ replacement therapy. Proceedings of the National Academy of Sciences, 2009, 106(32): p. 13475-13480.
7. Brooks, P., The burden of musculoskeletal disease—a global perspective. Clinical Rheumatology, 2006, 25(6): p. 778-781.
8. Rauh, J., et al., Bioreactor systems for bone tissue engineering. Tissue Eng Part B Rev, 2011, 17(4): p. 263-80.
9. Zhang, Y.-B., et al., Involvement of Oxidative Stress in Age—Related Bone Loss. Journal of Surgical Research, 2011, 169(1): p. e37-e42.
10. Sun, J.-S., et al., Vitamin-D Binding Protein Does Not Enhance Healing in Rat Bone Defects: A Pilot Study. Clinical Orthopaedics and Related Research®, 2009, 467(12): p. 3156-3164.
11. Huey, D. J., J. C. Hu, and K. A. Athanasiou, Unlike Bone, Cartilage Regeneration Remains Elusive. Science, 2012, 338(6109): p. 917-921.
12. Schubert, T., et al., Critical size bone defect reconstruction by an autologous 3D osteogenic-like tissue derived from differentiated adipose MSCs. Biomaterials, 2013, 34(18): p. 4428-4438.
13. Wang. W., et al., 3D spheroid culture system on micropatterned substrates for improved differentiation efficiency of multipoint mesenchymal stem cells. Biomaterials, 2009, 30(14): p. 2705-2715.
14. Lund, A. W., et al., The natural and engineered 3D microenvironment as a regulatory cue during stem cell fate determination. Tissue engineering. Part B, Reviews, 2009, 15(3): p. 371-380.
15. Rowley, J. A., G. Madlambayan, and D. J. Mooney, Alginate hydrogels as synthetic extracellular matrix materials. Biomaterials, 1999, 20(1): p. 45-53.
16. Draget, K. I., O. Smidsrød, and G. Skjåk-Braik, Alginates from Algae, in Biopolymers Online. 2005, Wiley-VCM Verlag GmbH & Co. KGaA.
17. Hwang, Y.-S., et al., The use of murine embryonic stem cells, alginate encapsulation, and rotary microgravity bioreactor in bone tissue engineering. Biomaterials, 2009, 30(4): p. 499-507.
18. Lee, K. Y. and D. J. Mooney, Alginate: Properties and biomedical applications. Progress in Polymer Science, 2012, 37(1): p. 106-126.
19. Pawar, S. N. and K. J. Edgar, Alginate derivatization: A review of chemistry, properties and applications. Biomaterials. 2012, 33(11): p. 3279-3305.
20. Sun, J.-Y., et al., Highly stretchable and tough hydrogels. Nature, 2012, 489(7414): p. 133-136.
21. Shull, K. R., Materials science: A hard concept in soft matter. Nature, 2012, 489(7414): p. 36-37.
22. Leslie, S. K., et al., Controlled release of rat adipose-thrived stem cells from alginate microbeads. Biomaterials. 2013, 34(33): p. 8172-8184.
23. Kuo, C. K. and P. X. Ma, Ionically crosslinked alginate hydrogels as scaffolds for tissue engineering: Part 1. Structure, gelation rate and mechanical properties. Biomaterials, 2001, 22(6): p. 511-521.
24. Awad. H. A., et al., Chondrogenic differentiation of adipose-derived adult stem cells in agarose, alginate, and gelatin scaffolds. Biomaterials, 2004, 25(16): p. 3211-3222.
25. Hunt, N. C., et al., Encapsulation of fibroblasts causes accelerated alginate hydrogel degradation. Acta Biomaterialia, 2010, 6(9): p. 3649-3656.
26. Higuchi, A., et al., Biomaterials for the Feeder-Free Culture of Human Embryonic Stem Cells and Induced Pluripotent Stem Cells. Chemical Reviews, 2011, 111(5): p. 3021-3035.
27. Shapiro, L. and S. Cohen, Novel alginate sponges for cell culture and transplantation. Biomaterials, 1997, 18(8): p. 583-590.
28. Ryan, J. M., et al., Mesenchymal stem cells avoid allogeneic rejection. J Inflamm (Lond), 2005, 2: p. 8.
29. Caplan, A. I., Adult mesenchymal stem cells for tissue engineering versus regenerative medicine. J Cell Physiol, 2007, 213(2): p. 341-7.
30. Lin. Y.-J., ct al., Chondrocytes culture in three-dimensional porous alginate scaffolds enhanced cell proliferation, matrix synthesis and gene expression. Journal of Biomedical Materials Research Part A, 2009, 88A(1): p. 23-33.
31. Chen, C.-Y., et al., 3D porous calcium-alginale scaffolds cell culture system improved human osteoblast cell dusters for cell therapy. Theranostics, 2015, 5(6): p. 643-655.
32. Oliver, L., et al., Distinct Roles of Bcl-2 anti Bcl-Xl in the Apoptosis of Human Bone Marrow Mesenchymal Stem Cells during Differentiation. PLoS ONE, 2011, 6(5): p. el9820.
33. Tzaphlidou, M. and V. Zaichick, Calcium, Phosphorus, calcium-phosphorus ratio in rib bone of healthy humans. Biological Trace Element Research, 2003. 93(1-3): p. 63-74.
34. Dorozhkin. S. V., Calcium orthopaosphates in nature, biology and medicine. Materials. 2009, 2(2): p. 399-498.
35. Freyman, T. M., et al., Fibroblast contraction of a collagen—GAG matrix. Biomaterials, 2001, 22(21): p. 2883-2891.
36. Hardy, R. and M. S. Cooper, Glucocorticoid-induced osteoporosis—a disorder of mesenchymal stromal cells? Frontiers in endocrinology, 2011, 2.
37. Wen, X., et al., Frequent epigenetic silencing of the bone morphogenetic protein 2 gene through methylation in gastric carcinomas. Oncogene, 2006, 25(18): p. 2666-2673.
38. Lavik, E. and R. Langer, Tissue engineering: current state and perspectives. Applied microbiology and biotechnology, 2004, 65(1): p. 1-8.
39. Enestvedt, C. K., et al., VEGF gene therapy augments localized angiogenesis and promotes anastomotic wound healing: a pilot study in a clinically relevant animal model. Journal of Gastrointestinal Surgery, 2008, 12(10): p. 1762-1772.
40. http://www.ebioscience.com/media/images/resources/knowledge-center/application/elisa/platinum-elisa-kits/platinum-elisa-plate.jpg.
41. http://www.nlm.nih.gov/medlineplus/ency/images/ency/fullsize/8745.jpg.
42. Mitsui, N., et al., Effect of compressive force on the expression of MMPs, PAs, and their inhibitors in osteoblastic Saos-2 cells. Life sciences, 2006, 79(6): p. 575-583.
43. Usta, S. N., et al., Chemically defined serum-free and xeno-free media for multiple cell lineages. Annals of Translational Medicine, 2014, 2(10): p. 97.
44. Pradel. W., et al., Cell passage and composition of culture medium effects proliferation and differentiation of human osteoblast-like cells from facial bone. Journal of physiology and pharmacology: an official journal of the Polish Physiological Society, 2008, 59 Suppl 5: p. 47-58.
45. K. Le Blanc, L. C. Davies, MSCs—cells with many sides, Cytotherapy 20(3) (2018) 273-278.
46. E. Santos, R. M. Hernandez, J. L. Pedraz, G. Orive, Novel advances in the design of three-dimensional bio-scaffolds to control cell fate: translation from 2D to 3D, Trends in biotechnology 30(6) (2012) 331-341.
47. P. Newman, Z. Lu, S. Roohani-Esfahani. T. L. Church. M. Biro. B. Davies, A. King, K. Mackenzie, A. Minett. H. Zreiqat. Porous and strong three-dimensional carbon nanotube coated ceramic scaffolds for tissue engineering. Journal of Materials Chemistry B 3(42) (2015) 8337-8347.
48. H. Tavassoli, S. N. Alhosseini. A. Tay, P. P. Chan, S. K. W. Oh, M. E. Warkiani, Large-scale Production of Stem Cells Utilizing Microcarriers: A Biomaterials Engineering Perspective from Academic Research to Commercialized Products, Biomaterials 181 (2018) 333-346.
49. K. M. Yamada. E. Cukierman, Modeling tissue morphogenesis and cancer in 3D, Cell 130(4) (2007) 601-610.
50. M. Martina, D. W. Hutmacher, Biodegradable polymers applied in tissue engineering research: a review. Polymer International 56(2) (2007) 145-157.
51. J. Yang, M. Yamato, C. Kohno. A Nishimoto, H. Sekine, F. Fukai, T. Okano, Cell sheet engineering: recreating tissues without biodegradable scaffolds, Biomaterials 26(33) (2005) 6415-6422.
52. L. da Silva Meirelles, A. I. Caplan, N. B. Nardi, In search of the in vivo identity of mesenchymal stem cells, Stem cells 26(9) (2008) 2287-2299.

What is claimed is:

1. A method of preparing a three-dimensional (3D) tissue-like implant containing specific cells, comprising
(a) seeding mesenchymal stem cells (MSCs) in an alginate scaffold to give a MSCs-alginate construct:
(b) transferring the MSCs-alginate construct into a perfusion bioreactor system; and
(c) incubating the MSCs-alginate construct in the perfusion bioreactor system under a condition that allows proliferation and differentiation of the MSCs toward the specific cells and formation of the 3D tissue -like implant which comprises the alginate scaffold embedded with a cell cluster comprising the MSCs and the specific cells.

2. The method of claim 1, wherein the condition comprises a culture medium comprising components to induce differentiation of the MSCs toward the specific cells.

3. The method of claim 1, wherein the specific cells are selected from the group consisting of osteo-like cells, chondro-like cells, muscle-like cells, neuron-like cells, adipo-like cells, bepato-like cells, lung-like cells, cardiac-like cells, fibroblast-like cells, and any combination of the above.

4. The method of claim 1, wherein the cell cluster forms a bone-like, cartilage-like, muscle-like, nerve-like, adipose-like, liver-like, lung-like, heart-like and/or blood vessels-like tissue.

5. The method of claim 1, wherein the cell cluster displays both a MSC surface marker and a differentiation marker of the specific cells.

6. The method of claim 1, wherein the cell cluster contains extracellular matrix (ECM) surrounding the cells.

7. The method of claim 1, further comprising (c) exposing the 3D tissue-like implant to a chelating agent to dissolve the scaffold to provide a scaffold-free 3D tissue-like implant.

8. The method of claim 1, further comprising (d) collecting the 3D tissue-like implant.

9. The method of claim 1, wherein the alginate scaffold is prepared by cross-linking of an alginate solution with a covalent crosslinking agent.

10. The method of claim 1, wherein tic MSCs are isolated from bone marrow, adipose tissue, muscle tissue, dental tissues, placenta, umbilical cord tissue, umbilical cord blood, peripheral blood.

11. The method of claim 1, wherein lie condition comprises an osteogenic medium to induce differentiation of the MSCs toward osteo-like cells.

12. The method of claim 11, wherein the osteogenic medium comprises a basic medium, a corticosteroid, and an inorganic phosphate source.

13. The method of claim 11, wherein the MSCs-alginate construct is cultured in the osteogenic medium within the bioreactor system for at least 1 day or more, 3 days or more, 7 days or more, 14 days or more, 21 days or more, 28 days or more.

14. The method of claim 11, wherein the cell cluster forms a bone-like tissue.

15. The method of claim 14, wherein the bone-like tissue includes both osteogenic and chondrogenic features.

16. The method of claim 14, wherein bone-like tissue contains an extracellular matrix (ECM) and/or a calcified area surrounding the cells.

17. The method of claim 14, wherein the bone-like tissue displays volumetric bone mineral density (vBMD) value from about 0.03 mg/cm³ to about 0.13 mg/cm³and/or Ca/P atomic ratio from about 1.85 to about 1.98.

18. The method of claim 14, wherein the bone-like tissue displays increasing volumetric bone mineral density (vBMD) value, increasing calcium ions and/or phosphorous ions, and/or increasing calcified areas overtime during the cultivation.

19. The method of claim 14, wherein the bone-like tissue includes hydroxyapatite (HAp).

20. The method of claim 14, wherein the bone-like tissue displays a MSC surface marker, a cartilage marker, an osteogenic marker/growth factor and/or an osteogenic cofactor/associated growth factor.

21. The method of claim 20, wherein
the MSC surface marker is selected from the group consisting of CD73, CD90, CD105 and any combination thereof;
the cartilage marker is secreted glycosaminoglycans (sGAG);
the osteogenic marker/growth factor is selected from the group consisting of alkaline phosphatase (ALP), osteocalcin (OCN); osteoprotegerin (OPG), bone morphogenetic protein-2 (BMP-2), tumor growth factor beta1 (TGFβ1), vascular endothelial growth factor A (VEGF-A) and any combination thereof; and
the osteogenic cofactor/associated growth factor is selected from the group consisting of sCD105, basic fibroblast growth factor (bFGF), stromal cell derived factor-1alpha (SDF-1α), vascular endothelial growth factor (VEGF) and any combination thereof.

22. The method of claim 14, wherein the osteogenic medium includes scrum.

23. A three-dimensional (3D) tissue-like implant or a pharmaceutical composition for transplanting into a subject in need, comprising a cell cluster comprising MSCs and specific cells differentiated therefrom, and optionally a pharmaceutically acceptable carrier.

24. The 3D tissue-like implant or the pharmaceutical composition of claim 23, wherein the cell cluster contains extracellular matrix (ECM) surrounding the cells.

25. The 3D tissue-like implant or the pharmaceutical composition of claim 23, wherein the cell cluster is embedded in an alginate scaffold.

26. The 3D tissue-like implant or the pharmaceutical composition of claim 23, which does not include a scaffold.

27. The 3D tissue-like implant or the pharmaceutical composition of claim 23, wherein the specific cells are osteo-like cells and the cell cluster forms a bone-like tissue.

28. The 3D tissue-like implant or the pharmaceutical composition of claim 27,
wherein the bone-like tissue includes both osteogenic and chondrogenic features;
wherein the cell cluster surrounds with extracellular matrix (ECM) and/or calcified areas;
wherein the bone-like tissues display volumetric bone mineral density (vBMD) value from about 0.03 mg/cm³ to about 0.13 mg/cm³ and/or Ca/P atomic ratio from about 1.85 to about 1.98;
wherein the bone-like tissues include hydroxyapatite (HAp); and/or
wherein the bone-like tissues display a MSC surface marker, a cartilage marker, an osteogenic marker/growth factor and/or an osteogenic cofactor/associated growth factor.

29. The 3D tissue-like implant or the pharmaceutical composition of claim 28.
wherein the MSC surface marker is selected from the group consisting of CD73, CD90, CD105 and any combination thereof;
wherein the cartilage marker is secreted glycosaminoglycan (sGAG);
wherein the osteogenic marker/growth factor is selected from the group consisting of alkaline phosphatase (ALP), osteocalcin (OCN); osteoprotegerin (OPG), bone morphogenetic protein-2 (BMP-2), tumor growth factor beta 1 (TGFβ1), vascular endothelial growth factor A (VEGF-A) and any combination thereof; and/or
wherein the osteogenic cofactor/associated growth factor is selected from the group consisting of sCD105, basic fibroblast growth factor (bFGF), stromal cell derived factor-1alpha (SDF-1α), vascular endothelial growth factor (VEGF) and any combination thereof.

30. A three-dimensional (3D) tissue-like implant for transplanting into a subject in need prepared by a method of claim 1.

31. A method for repairing a bone defect in a patient in need, comprising placing the 3D-tissue like implant or the pharmaceutical composition of claim 27 in the patient at a bone defective site.

32. A method for repairing a bone defect in a recipient patient in need, comprising
(i) providing a three-dimensional (3D) bone-like implant which is prepared by a method comprising (a) seeding mesenchymal stem cells (MSCs) in an alginate scaffold to give a MSCs-alginate construe:; (b) transferring the MSCs-alginate construct into a perfusion bioreactor system for cultivation under a condition that allows proliferation and differentiate of the MSCs toward osteo-like cells and formation of the 3D bone-like implant comprising the alginate scaffold embedded with a cell cluster comprising the MSCs and the osteo-like cells; (c) optionally exposing the 3D bone-like implant to a chelating agent to dissolve the scaffold to provide a scaffold-free 3D bone-like implant: and (d) collecting the 3D bone-like implant;
(ii) placing die 3D-bone like implant to the patient at a bone defective site at an amount effective to repair the bone defect.

33. The method of claim 32, wherein the MSCs are isolated from bone marrow, adipose tissue, muscle tissue, dental tissues, placenta, umbilical cord tissue, umbilical cord blood, peripheral blood of a donor subject.

34. The method of claim 33, wherein the donor subject is the recipient subject.

35. A method for treating a defect in a recipient patient in need, comprising placing a 3D-tissue-like implant or a pharmaceutical composition of claim 23 to the patient at a defective site at an amount effective to treat the defect.
